(19) United States
(12) Patent Application Publication
(10) Pub. No.: US 2026/0361115 A1
(43) Pub. Date: Nov. 12, 2026

(54) BATTERY PRECONDITIONING FOR A WEARABLE MONITOR

(71) Applicant: Whoop, Inc., Boston, MA (US)

(72) Inventors: Abhineet Nigam, Somerville, MA (US); Elizabeth Anne Sorenti, Medford, MA (US); Genevieve Rae Senecal, Cambridge, MA (US); Jacob Sheridan Walsh, Somerville, MA (US)

(21) Appl. No.: 19/669,811

(22) Filed: May 6, 2026

Related U.S. Application Data
(60) Provisional application No. 63/801,472, filed on May 7, 2025.

(51) Int. Cl. H02J 7/90; A61B 5/00; G06F 1/16; H01M 10/48; H01M 10/615; H01M 10/623; H01M 10/633; H01M 10/637; H01M 10/653; H01M 50/247; H02J 7/00

(52) U.S. Cl. CPC H02J 7/977; A61B 5/6831; G06F 1/163; H01M 10/486; H01M 10/488; H01M 10/615; H01M 10/623; H01M 10/633; H01M 10/637; H01M 10/653; H01M 50/247; H02J 7/855; A61B 2560/0214; H01M 2220/30

(57) ABSTRACT

Heat sources within a wearable device, such as induction coils for wireless charging or NFC communications, light emitting diodes, and so forth, are used to pre-heat an internal battery of a wearable device under certain conditions (such as ambient cool temperatures) to improve battery performance.

### RELATED APPLICATIONS

This application claims the benefit of U.S. Prov. App. No. 63/801,472 filed on May. 7, 2025, the entire content of which is hereby incorporated by reference.

### BACKGROUND

Battery chemistry can make batteries in consumer devices highly sensitive to ambient temperatures, particularly in outdoor environments where temperature fluctuations are common. Users may experience reduced battery life, slower charging times, and decreased device performance when using their electronics in cold conditions. There remains a need for battery-powered electronics such as wearable devices that thermally precondition batteries for improved cold-weather performance.

### SUMMARY

Heat sources within a wearable device, such as induction coils for wireless charging or NFC communications, light-emitting diodes, and so forth, are used to pre-heat an internal battery of a wearable device under certain conditions (such as ambient cool temperatures) to improve battery performance.

A wearable device disclosed herein includes physiological monitoring hardware; a housing; a rechargeable battery disposed within the housing; an induction coil positioned in the housing proximate to the rechargeable battery; an LED positioned in the housing proximate to the rechargeable battery; a temperature sensor configured to monitor a temperature of the rechargeable battery; and a control circuit operatively connected to the induction coil, the LED, and the temperature sensor, wherein the control circuit is configured to: select at least one of the induction coil and the LED as a selected heating element to heat the rechargeable battery in response to the temperature of the rechargeable battery failing to meet a predetermined threshold, operate the selected heating element to increase the temperature of the rechargeable battery, and initiate a discharge of the rechargeable battery to operate the physiological monitoring hardware of the wearable device for acquisition of physiological data in response to the temperature falling within a predetermined temperature range.

The wearable device may include a thermally conductive material positioned between at least one of the induction coil or the LED and the rechargeable battery to enhance an efficiency of heat transfer to the rechargeable battery. The control circuit may be further configured to select between activating the induction coil or the LED based on an available power source in the wearable device. The control circuit may be configured to activate the induction coil as the selected heating element when external power may be available through a charging port and activate the LED as the selected heating element when operating the device from the rechargeable battery. The control circuit may be configured to implement a graduated heating process by incrementally increasing power to the selected heating element as the temperature of the rechargeable battery increases toward the predetermined temperature range. The control circuit may be configured to deactivate the selected heating element upon reaching the predetermined temperature range.

In another aspect, a battery preconditioning system for a wearable device, as disclosed herein, includes: a rechargeable battery; a heating element positioned proximate to the rechargeable battery; a temperature sensor configured to monitor a temperature of the rechargeable battery; and a control circuit operatively connected to the heating element and the temperature sensor, wherein the control circuit may be configured to: activate the heating element to heat the rechargeable battery in response to the temperature of the rechargeable battery failing to meet a predetermined threshold, and initiate charging or discharging of the rechargeable battery in response to the temperature of the rechargeable battery falling within a predetermined temperature range.

The heating element may include an induction coil. The induction coil may be configured to generate heat without initiating a charging process. The heating element may include an LED configured to generate heat at a low power setting. The system may include a thermally conductive material positioned between the heating element and the rechargeable battery to enhance heat transfer efficiency. The control circuit may be configured to deactivate the heating element in response to the temperature reaching the predetermined temperature range. The wearable device may include a wearable fitness monitor. The temperature sensor may include a thermistor.

In another aspect, a method of preconditioning a rechargeable battery in a device, as disclosed herein, includes: detecting a temperature associated with the rechargeable battery using a temperature sensor; determining that the temperature fails to meet a predetermined threshold; in response to the temperature failing to meet the predetermined threshold, activating a heating element of a parasitic heat source proximate to the rechargeable battery to heat the rechargeable battery; monitoring the temperature as the rechargeable battery may be heated with the heating element; and in response to the temperature falling within a predetermined temperature range, initiating a use of the rechargeable battery.

The parasitic heat source of the device may have a function on the device including one or more of physiological sensing, environmental monitoring, data acquisition, power transfer, and communications with a remote device. The heating element may include an induction coil, and activating the heating element may include energizing the induction coil to generate heat without initiating a charging process for the rechargeable battery. The heating element may include an LED, and activating the heating element may include operating the LED to generate heat for the rechargeable battery. The method may include gradually increasing a power to the LED as the temperature of the rechargeable battery approaches the predetermined temperature range. The device may include an LED and an induction coil, the method further comprising selecting between the LED and the induction coil for use as the heating element based on a rate of temperature increase required to reach the predetermined temperature range within a predetermined time period.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, with emphasis instead being placed upon illustrating the principles of the devices, systems, and methods described herein. In the drawings, like reference numerals generally identify corresponding elements.

FIG. 1 shows a physiological monitoring device.

FIG. 2 shows a physiological monitoring system.

FIG. 3 shows a sensing system.

FIG. 4A shows examples of physiological monitoring devices.

FIG. 4B shows examples of physiological monitoring devices.

FIG. 4C shows examples of physiological monitoring devices.

FIG. 5 shows a smart garment system.

FIG. 6 shows a system for battery pre-heating.

FIG. 7 is a flow chart of a method for thermal conditioning during battery charging.

FIG. 8 is a flow chart of a method for battery preheating.

### DESCRIPTION

Embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better describe the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” “above,” “below,” and the like, are words of convenience and are not to be construed as limiting terms unless specifically stated to the contrary.

The term “user” as used herein, refers to any type of animal, human or non-human, whose physiological information may be monitored using an exemplary wearable physiological monitoring device and/or system.

The term “continuous,” as used herein in connection with heart rate data, refers to the acquisition of heart rate data at a sufficient frequency to enable detection of individual heartbeats, and also refers to the collection of heart rate data over extended periods, such as an hour, a day or more (including acquisition throughout the day and night), etc. More generally, with respect to physiological signals that might be monitored by a wearable device, “continuous” or “continuously” will be understood to mean continuously at a rate and duration suitable for the intended time-based processing, and physically at an inter-periodic rate (e.g., multiple times per heartbeat, respiration, and so forth) sufficient for resolving the desired physiological characteristics, such as heart rate, heart rate variability, heart rate peak detection, pulse shape, and so forth. Continuous monitoring should also be understood to include periodic sampling at any suitable interval, duration, and frequency. Thus, for example, continuous monitoring may include measuring a user's body temperature once every ten minutes, or monitoring heart activity by alternately sampling the heart rate for a minute and then pausing sampling for a minute, e.g., to conserve power or memory at times when the measured heart rate indicates that the user is at rest. Sampling may also be dynamic based on sensor input, for example, increasing the sampling rate when signal variability increases, or during periods of relatively higher motion, or based on user input.

At the same time, continuous monitoring is not intended to exclude ordinary data acquisition interruptions, such as temporary displacement of monitoring hardware due to sudden movements, changes in external lighting, loss of electrical power, physical manipulation and/or adjustment by a wearer, physical displacement of monitoring hardware due to external forces, and so forth. It will also be noted that heart rate data or a monitored heart rate, in this context, may more generally refer to raw sensor data, such as optical intensity signals, or processed data therefrom, such as heart rate data, signal peak data, heart rate variability data, or any other physiological or digital signal suitable for recovering heart rate information as contemplated herein. Furthermore, such heart rate data may generally be captured over some historical period that can be subsequently correlated to various other data or metrics related to, e.g., sleep states, recognized exercise activities, resting heart rate, maximum heart rate, and so forth.

The term “computer-readable medium,” as used herein, refers to any non-transitory storage media, such as storage hardware, storage devices, computer memory that may be accessed by a controller, a microcontroller, a microprocessor, a computational system, or the like, or any other module or component of a computational system to encode thereon computer-executable instructions, software programs, and/or other data. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), virtual or physical computer system memory, physical memory hardware, such as random access memory (such as DRAM, SRAM, EDO RAM), and so forth. Although not depicted, any of the devices or components described herein may include a computer-readable medium or other memory for storing program instructions, data, and the like.

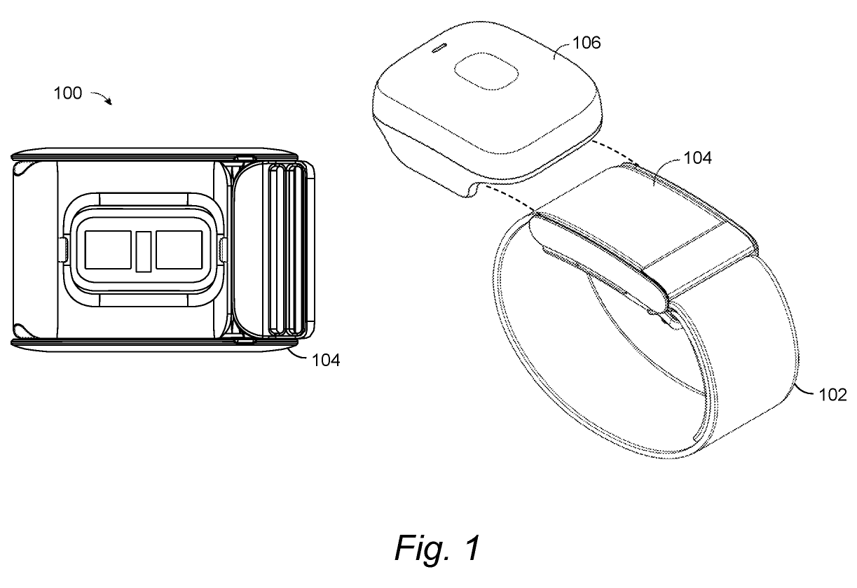

FIG. 1 shows a physiological monitoring system. The system 100 may include a wearable monitor 104 that is configured for physiological monitoring. The system 100 may also include a removable and replaceable battery 106 for recharging the wearable monitor 104. The wearable monitor 104 may include a strap 102 or other retaining system(s) for securing the wearable monitor 104 in a position on a wearer’s body for the acquisition of physiological data as described herein. For example, the strap 102 may include a slim elastic band formed of any suitable elastic material such as rubber or a woven polymer fiber such as a woven polyester, polypropylene, nylon, spandex, and so forth. The strap 102 may be adjustable to accommodate different wrist sizes, and may include any latches, hasps, or the like to secure the wearable monitor 104 in an intended position for monitoring a physiological signal. While a wrist-worn device is depicted, it will be understood that the wearable monitor 104 may be configured for positioning in any suitable location on a user’s body, based on the sensing modality and the nature of the signal to be acquired. For example, the wearable monitor 104 may be configured for use on a wrist, a forearm, an ankle, a lower leg, a bicep, a chest, side torso, back, a gluteus, behind the ear, forehead, or any other suitable location(s), and the strap 102 may be, or may include, a waistband or other elastic band or the like within an article of clothing or accessory. In another aspect, the wearable monitor 104 may be configured as a ring, earring, stick-on, clip-on, head-mounted (e.g., glasses or goggles), or other article of clothing or accessory that can be worn by a user, and that contains suitable instrumentation, memory, and/or processing for physiological monitoring as described herein. The wearable monitor 104 may also or instead be structurally configured for placement on or within a garment, e.g., permanently or in a removable and replaceable manner. To that end, the wearable monitor 104 may be shaped and sized for placement within a pocket, slot, and/or other housing that is coupled to or embedded within a garment. In such configurations, the pocket or other retaining arrangement on the garment may include sensing windows or the like so that the wearable monitor 104 can operate while placed for use in the garment. United States Pat. No. 11,185,292 and U.S. Pat. Pub. No. 2024/0106283 describe non-limiting example embodiments of suitable wearable monitors 104, and are incorporated herein by reference in their entirety. And while the present disclosure may refer to a wrist-worn wearable or other wearable, it should be understood that any of the other locations or forms described herein are also included unless expressly stated to the contrary or otherwise clear from the context.

The system 100 may include any hardware components, subsystems, and the like to support various functions of the wearable monitor 104, such as data collection, processing, display, and communications with external resources. For example, the system 100 may include hardware for a heart rate monitor using, e.g., photoplethysmography, electrocardiography, or any other technique(s). The system 100 may be configured such that, when the wearable monitor 104 is placed for use about a wrist (or at some other body location), the system 100 initiates acquisition of physiological data from the wearer. In some embodiments, the pulse or heart rate may be acquired optically based on a light source (such as light-emitting diodes (LEDs)) and optical detectors in the wearable monitor 104. The LEDs may be positioned to direct illumination toward the user’s skin, and optical detectors such as photodiodes may be used to capture illumination intensity measurements indicative of illumination from the LEDs that is reflected and/or transmitted by or through the wearer’s skin, or depending on the configuration, through capillaries or arteries.

The system 100 may be configured to record other physiological and/or biomechanical parameters, including, but not limited to, skin temperature (using a thermometer), galvanic skin response (using a galvanic skin response sensor), motion (using one or more multi-axis accelerometers and/or gyroscopes), blood pressure (via physical pressure measurements or other means), sound, electrocardiograms, and the like, as well as environmental or contextual parameters such as ambient light, ambient temperature, humidity, time of day, location, and so forth. For example, the wearable monitor 104 may include sensors such as accelerometers and/or gyroscopes for motion detection, sensors for environmental temperature sensing, sensors to measure electrodermal activity (EDA), sensors to measure galvanic skin response (GSR) sensing, and so forth. The system 100 may also or instead include other systems or subsystems supporting additional functions of the wearable monitor 104. For example, the system 100 may include communications systems to support, e.g., near-field communications, proximity sensing, touch sensing (e.g., via capacitive or resistive sensors), Bluetooth communications, Wi-Fi communications, cellular communications, satellite communications, and so forth. The wearable monitor 104 may also or instead include components such as a Global Positioning System (GPS), a display and/or user interface, a clock and/or timer, and so forth.

The wearable monitor 104 may include one or more sources of battery power, such as a first battery within the wearable monitor 104 and a second battery 106 that is removable from and replaceable to the wearable monitor 104 in order to recharge the battery in the wearable monitor 104. The wearable monitor 104 may also or instead include systems for energy harvesting via, e.g., kinetic energy capture, ambient electromagnetic radiation capture, solar/optical energy capture, and so forth, as well as systems for short and/or medium-range wireless energy transfer to receive power from nearby wireless power sources. Also or instead, the system 100 may include a plurality of wearable monitors 104 (and/or other physiological monitors) that can share battery power or provide power to one another, e.g., using a garment power infrastructure, wireless power sharing network, or the like. The system 100 may perform numerous functions related to continuous monitoring, such as automatically detecting when the user is asleep, awake, exercising, and so forth, and such detections may be performed locally at the wearable monitor 104 or at a remote service such as a mobile device or cloud computing resource coupled in a communicating relationship with the wearable monitor 104 and receiving data therefrom. In general, the system 100 may support continuous, independent monitoring of a physiological signal such as a heart rate, and the underlying acquired data may be stored on the wearable monitor 104 for an extended period until it can be uploaded to a remote processing resource for more computationally complex analysis. In one aspect, the wearable monitor 104 may be a wrist-worn photoplethysmography device, although other form factors are also or instead possible as described herein, such as a ring, a bicep band, a calf band, an elastic band in a garment, a patch, a clip-on device, and so forth.

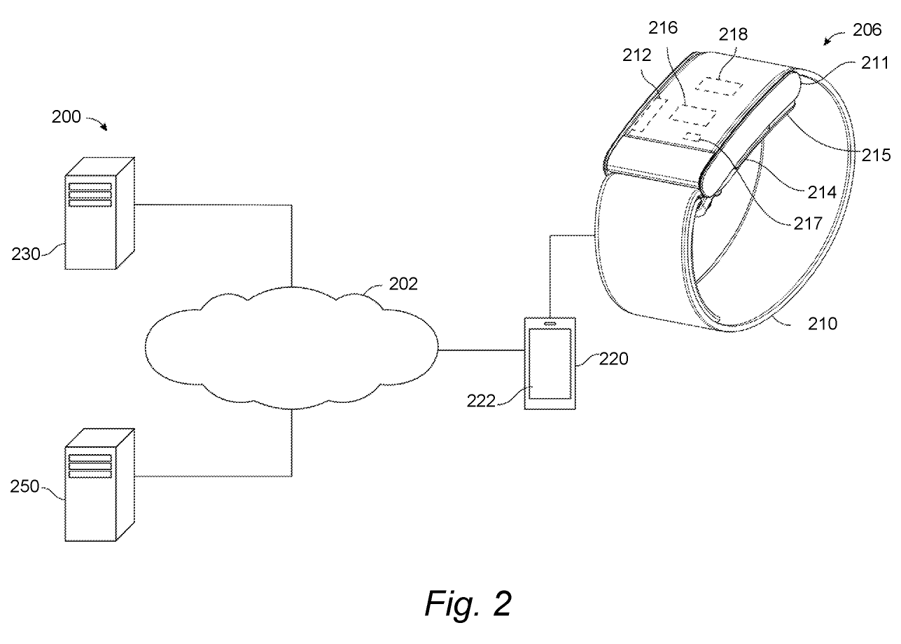

FIG. 2 illustrates a physiological monitoring system. More specifically, FIG. 2 illustrates a physiological monitoring system 200 that may be used with any of the methods or devices described herein. In general, the system 200 may include a physiological monitor 206, a user device 220, a remote server 230 with a remote data processing resource (such as any of the processors or processing resources described herein), and one or more other resources 250, all of which may be interconnected through a data network 202.

The data network 202 may be any of the data networks described herein. For example, the data network 202 may be any network(s) or internetwork(s) suitable for communicating data and information among participants in the system 200. This may include public networks such as the Internet, private networks, telecommunications networks such as the Public Switched Telephone Network or cellular networks using third generation (e.g., 3G or IMT-200), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 200. This may also include local or short-range communications infrastructure suitable, e.g., for coupling the physiological monitor 206 to the user device 220, or otherwise supporting communication with local resources. By way of non-limiting examples, short-range communications may include Wi-Fi communications, Bluetooth communications, infrared communications, near-field communications, communications with RFID tags or readers, and so forth.

The physiological monitor 206 may, in general, be any physiological monitoring device or system, such as any of the wearable monitors or other monitoring devices or systems described herein. In one aspect, the physiological monitor 206 may be a wearable physiological monitor shaped and sized to be worn on a wrist or other body location. The physiological monitor 206 may include a wearable housing 211, a network interface 212, one or more sensors 214, one or more light sources 215, a processor 216, a haptic device 217 or other user input/output hardware, a memory 218, and a strap 210 for retaining the physiological monitor 206 in a desired location on a user. In one aspect, the physiological monitor 206 may be configured to acquire heart rate data and/or other physiological data from a wearer in an intermittent or substantially continuous manner. In another aspect, the physiological monitor 206 may be configured to support extended, continuous acquisition of physiological data, e.g., for several days, a week, or more.

In one aspect, the physiological monitor 206 may be configured to continuously or intermittently acquire, store, and/or process physiological data at a frequency sufficient to resolve cardiac activity, including a heart rate and heart rate variability. This may, for example, usefully include sampling rates of 50 Hz (20 ms) or greater on one or more optical channels, as well as synchronized acquisition of temperature data, motion data, and the like. As a significant advantage, this rate of data acquisition can facilitate real-time or near real-time delivery of metrics such as any of the physiological metrics described herein, as well as accurate peak detection, resolution of heart rate variability (typically reported in milliseconds), and extraction of other cardiac metrics and so forth. Similarly, the availability of computational resources, either local or remote, to perform signal processing, embedding, foundation model inferencing, and the like can support real-time coaching or the like to enable timely, actionable user feedback based on the data acquired with the physiological monitor.

The network interface 212 of the physiological monitor 206 may be configured to couple the physiological monitor 206 to one or more other components of the system 200 in a communicating relationship, either directly, e.g., through a cellular data connection or the like, or indirectly through a short-range wireless communications channel coupling the physiological monitor 206 locally to a wireless access point, router, computer, laptop, tablet, cellular phone, or other device that can locally process data, and/or relay data from the physiological monitor 206 to the remote server 230 or other resource(s) 250 as necessary or helpful for acquiring and processing data from the physiological monitor 206. The network interface 212 may also or instead facilitate connections among multiple wearable devices, power sources, and the like, e.g., in a wearable device area network or other multi-device monitoring infrastructure.

The one or more sensors 214 may include any of the sensors described herein, or any other sensors or sub-systems suitable for physiological monitoring or supporting functions. By way of example and not limitation, the one or more sensors 214 may include one or more of a light source (including, e.g., LEDs or other wavelength-specific sources of green light, red light, infrared light, and so forth, as well as broadband illumination), an optical sensor, an accelerometer, a gyroscope, a temperature sensor, a galvanic skin response sensor, a capacitive sensor, a resistive sensor, an environmental sensor (e.g., for measuring ambient temperature, humidity, lighting, and the like), a geolocation sensor, and so forth. The one or more sensors 214 may also or instead include sensors (and accompanying hardware/software) for, e.g., a Global Positioning System, a proximity sensor, an RFID tag reader, an RFID tag, a temporal sensor, an electrodermal activity sensor, an electrocardiogram, a pressure sensor, an acoustic sensor (e.g., a microphone), a camera (e.g., visible light and/or infrared), and the like. The one or more sensors 214 may be disposed in the wearable housing 211, or otherwise positioned and configured for physiological monitoring or other functions described herein. In one aspect, the one or more sensors 214 include a light detector configured to provide light intensity data to the processor 216 (or to the remote server 230) for calculating a heart rate and a heart rate variability. The one or more sensors 214 may also or instead include an accelerometer, gyroscope, and the like configured to provide motion data to the processor 216, e.g., for detecting activities such as a sleep state, a resting state, a waking event, exercise, and/or other user activity. In an implementation, the one or more sensors 214 may include a sensor to measure a galvanic skin response of the user. The one or more sensors 214 may also or instead include electrodes or the like for capturing electronic signals, e.g., to obtain an electrocardiogram and/or other electrically-derived physiological measurements.

The processor 216 and memory 218 may be any of the processors and memories described herein. In one aspect, the memory 218 may store physiological data obtained by monitoring a user with the one or more sensors 214, and/or any other sensor data, program data, or other data useful for operation of the physiological monitor 206 or other components of the system 200. It will be understood that, while only the memory 218 on the physiological monitor is illustrated, any other device(s) or components of the system 200 may also or instead include a memory to store program instructions, raw data, processed data, user inputs, and so forth. In one aspect, the processor 216 of the physiological monitor 206 may be configured to obtain heart rate data from the user, such as heart rate data including or based on the raw data from the sensors 214. The processor 216 may also or instead be configured to determine, or assist in a determination of, a condition of the user related to, e.g., health, fitness, strain, recovery, sleep, or any of the other conditions described herein.

The one or more light sources 215 may be coupled to the wearable housing 211 and controlled by the processor 216. At least one of the light sources 215 may be directed toward the skin of a user adjacent to the wearable housing 211. Light from the light source 215, or more generally, light at one or more wavelengths of the light source 215, may be detected by one or more of the sensors 214, and processed by the processor 216 as described herein.

The system 200 may further include a remote data processing resource executing on a remote server 230. The remote data processing resource may include any of the processors and related hardware described herein, and may be configured to receive data transmitted from the memory 218 of the physiological monitor 206, and to process the data to detect or infer physiological signals of interest such as heart rate, heart rate variability, respiratory rate, pulse oxygen, blood pressure, and so forth. The remote server 230 may also or instead evaluate a condition of the user such as a recovery state, sleep state, exercise activity, exercise type, sleep quality, daily activity strain, and any other health or fitness conditions that might be detected based on such data.

The system 200 may include one or more user devices 220, which may work together with the physiological monitor 206, e.g., to provide a display, or more generally, user input/output, for user data and analysis, and/or to provide a communications bridge from the network interface 212 of the physiological monitor 206 to the data network 202 and the remote server 230. For example, the physiological monitor 206 may communicate locally with a user device 220, such as a smartphone of a user, via short-range communications, e.g., Bluetooth, or the like, for the exchange of data between the physiological monitor 206 and the user device 220, and the user device 220 may in turn communicate with the remote server 230 via the data network 202 in order to forward data from the physiological monitor 206 and to receive analysis and results from the remote server 230 for presentation to the user. In one aspect, the user device(s) 220 may support physiological monitoring by processing or pre-processing data from the physiological monitor 206 to support extraction of heart rate or heart rate variability data from raw data obtained by the physiological monitor 206. In another aspect, computationally intensive processing may advantageously be performed at the remote server 230, which may have greater memory capabilities and processing power than the physiological monitor 206 and/or the user device 220.

The user device 220 may include any suitable computing device(s) including, without limitation, a smartphone, a desktop computer, a laptop computer, a network computer, a tablet, a mobile device, a portable digital assistant, a cellular phone, a portable media or entertainment device, or any other computing devices described herein, including, e.g., supplemental wearable devices and/or computers. The user device 220 may provide a user interface 222 for access to data and analysis by a user, and/or to support user control of operation of the physiological monitor 206. The user interface 222 may be maintained by one or more applications executing locally on the user device 220, or the user interface 222 may be remotely served and presented on the user device 220, e.g., from the remote server 230 or the one or more other resources 250.

In general, the remote server 230 may include data storage, a network interface, and/or other processing circuitry. The remote server 230 may process data from the physiological monitor 206 and perform physiological and/or health monitoring/analyses or any of the other analyses described herein, (e.g., analyzing sleep, determining strain, assessing recovery, and so on), and may host a user interface for remote access to this data, e.g., from the user device 220. The remote server 230 may include a web server or other programmatic front end that facilitates web-based access by the user devices 220 or the physiological monitor 206 to the capabilities of the remote server 230 or other components of the system 200.

The system 200 may include other resources 250, such as any resources that can be usefully employed in the devices, systems, and methods as described herein. For example, these other resources 250 may include other data networks, databases, processing resources, cloud data storage, data mining tools, computational tools, data monitoring tools, algorithms, and so forth. In another aspect, the other resources 250 may include one or more administrative or programmatic interfaces for human actors such as programmers, researchers, annotators, editors, analysts, coaches, and so forth, to interact with any of the foregoing. The other resources 250 may also or instead include any other software or hardware resources that may be usefully employed in the networked applications as contemplated herein. For example, the other resources 250 may include payment processing servers or platforms used to authorize payment for access, content, or option/feature purchases. In another aspect, the other resources 250 may include certificate servers or other security resources for third-party verification of identity, encryption or decryption of data, and so forth. In another aspect, the other resources 250 may include a desktop computer or the like co-located (e.g., on the same local area network with, or directly coupled to through a serial or USB cable) with a user device 220, wearable strap 210, or remote server 230. In this case, the other resources 250 may provide supplemental functions for components of the system 200 such as firmware upgrades, user interfaces, and storage and/or pre-processing of data from the physiological monitor 206 before transmission to the remote server 230.

The other resources 250 may also or instead include one or more web servers that provide web-based access to and from any of the other participants in the system 200. While depicted as a separate network entity, it will be readily appreciated that the other resources 250 (e.g., a web server) may also or instead be logically and/or physically associated with one of the other devices described herein, and may, for example, include or provide a user interface 222 for web access to the remote server 230 or a database or other resource(s) to facilitate user interaction through the data network 202, e.g., from the physiological monitor 206 or the user device 220.

In another aspect, the other resources 250 may include fitness equipment or other fitness infrastructure. For example, a strength training machine may automatically record repetitions and/or added weight during repetitions, which may be wirelessly accessible by the physiological monitor 206 or some other user device 220. More generally, a gym may be configured to track user movement from machine to machine, and report activity from each machine in order to track various strength training activities in a workout. The other resources 250 may also or instead include other monitoring equipment or infrastructure. For example, the system 200 may include one or more cameras to track motion of free weights and/or the body position of the user during repetitions of a strength training activity or the like, and/or the cameras may be integrated into the physiological monitor 206 or other user device 220. Similarly, a user may wear, or have embedded in clothing, tracking fiducials such as visually distinguishable objects for image-based tracking, or radio beacons or the like for other tracking. In another aspect, weights may themselves be instrumented, e.g., with sensors to record and communicate detected motion, and/or beacons or the like to self-identify type, weight, and so forth, in order to facilitate automated detection and tracking of exercise activity with other connected devices.

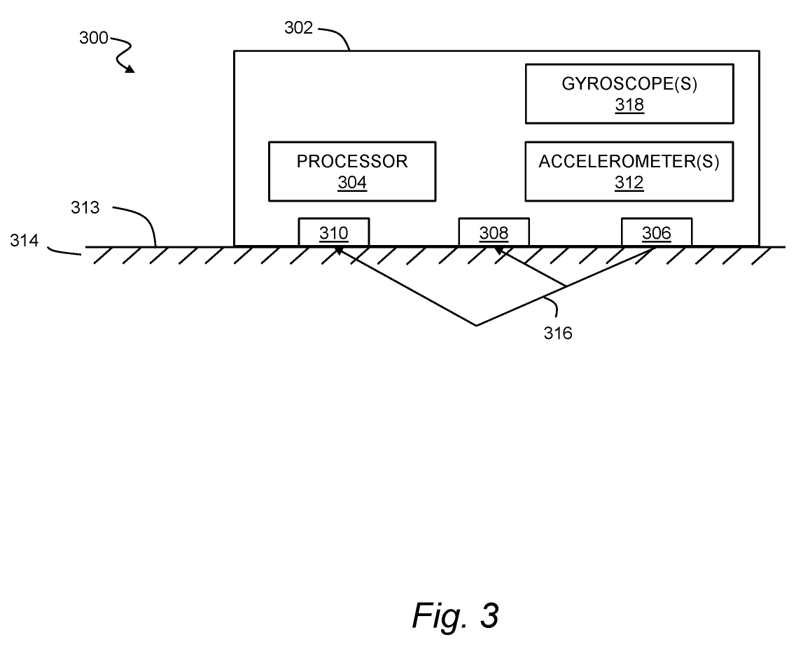

FIG. 3 shows a sensing system. In general, the system 300 may include a physiological monitor 302 with a processor 304, a light source 306, a first sensor 308 (e.g., a first photodetector), a second sensor 310 (e.g., a second photodetector), one or more accelerometers 312, one or more gyroscopes 318, and any other hardware or other components and systems suitable for physiological monitoring as described herein. The physiological monitor 302 may be positioned for use against a surface 313 of the skin 314 of a user where the light source 306 and sensors 308, 310 can contact the skin 314 for acquisition of physiological data. Although not depicted, it will be understood that the physiological monitor 302 may generally be retained in position using any of the straps, garments, patches, bands, clamps, clips, or the like described herein, and/or integrated into other wearable garments, accessories, and the like, such as audio earbuds, earrings or similar, glasses and/or other eyewear, a ring, a headband, and so forth.

The processor 304 may be any microprocessor, microcontroller, application-specific integrated circuit, or other processing circuitry or combination of the foregoing suitable for controlling operation of the physiological monitor and acquiring physiological data.

The light source 306 may include one or more light-emitting diodes or other sources of illumination, and may be positioned within the physiological monitor 302 such that, when the physiological monitor 302 is placed for use on the skin 314, the light source 306 directs illumination toward the skin 314 and the illumination is reflected back toward the sensors 308, 310 as indicated by arrows 316 (or transmitted through the tissue to one or more opposing sensors), where the intensity can be measured. In one aspect, the light source 306 may include light-emitting diodes that emit light in the green, red, infrared, near-infrared, or other suitable wavelength ranges, which can provide desired light transmission through human skin, facilitating low-power transmission of measurable illumination to the sensors 308, 310, although other illumination sources and wavelengths may also or instead be used.

The sensors 308, 310 may be oriented to contact the skin 314 when the physiological monitor 302 is placed for use on this skin 314, and positioned so that the sensors 308, 310 can capture illumination reflected and/or transmitted by the skin from the light source 306. In general, the sensors 308, 310 may include photodiodes, photodetectors, or any other sensor(s) responsive to illumination from the light source 306. This may include broadband optical sensors, narrowband optical sensors, filtered sensors, or the like. In general, a first sensor 308 may be positioned closer to the light source 306 than a second sensor 310 to facilitate detection of differential intensity in the measured wavelength(s). For example, the first sensor 308 may be positioned 1–4 millimeters from the light source 306 and the second sensor 310 may be positioned 2–8 millimeters from the light source, or about twice as far as the first sensor 308 from the light source 306.

Other spacings may also or instead be used depending on, e.g., the intensity of the light source 306, the sensitivity of the sensors 308, 310, the contact force of the physiological monitor 302 on the skin 314, the degree of incursion of ambient light, the physiological measurements/properties of interest, and so forth. In one aspect, the sensors 308, 310 may be linearly arranged in a straight line away from the light source 306. While this provides consistency in comparative measurements, it is not strictly required, and the sensors may be displaced in any of a number of directions away from the light source 306 provided they both contact the skin 314 in a manner that permits capture of light through the skin 314 from the light source 306. In another aspect, the physiological monitor 302 may include one or more other light sources and/or light sensors, which may be arranged to improve accuracy and/or provide redundancy for the contact detection, or to support other measurements such as oxygenation or skin thickness. This may include light sources/sensors using different ranges of wavelengths, different patterns of illumination, and so forth. In another aspect, two sensors may be positioned at different distances from a perimeter of the physiological monitor 302 so that the sensors can acquire differential intensity values for ambient light incident on the skin and transmitted through the skin to the sensors.

In operation, the processor 304 may acquire raw intensity data from the sensors 308, 310, and perform local calculations such as pre-processing raw data for heart rate measurements, or evaluating whether the physiological monitor 302 is properly placed for use on the skin 314.

The accelerometer 312 may include, e.g., one or more single-axis or multi-axis accelerometers, which may usefully measure motion of the physiological monitor 302 to support calculations such as automated activity detection, device on/off evaluation, degree of musculoskeletal activation, and so forth. Other motion and orientation sensing hardware—such as one or more gyroscopes 318, inertial motion sensors, and/or other micro-electromechanical system (MEMS) sensors—may also or instead be used for these purposes. More generally, the physiological monitor 302 may include any additional components, subsystems, and the like suitable for supporting various modes of physiological monitoring and contextual data acquisition as described herein.

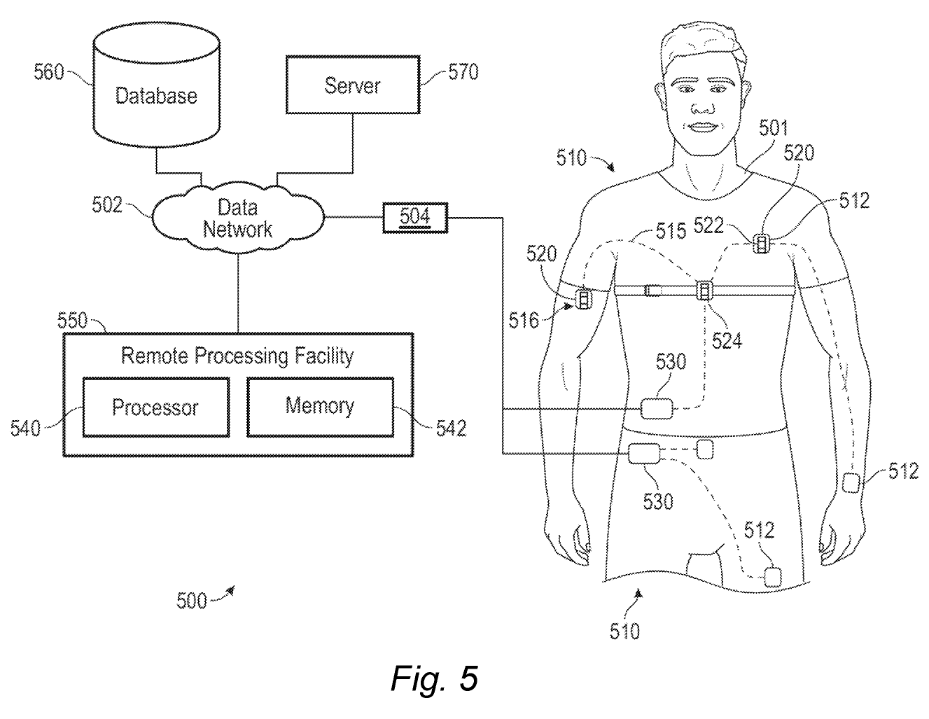

The physiological monitors described herein—e.g., in the systems 100, 200, 300 described above or elsewhere herein—may be provided in one or more different form factors. That is, although a wrist-worn device is illustrated in FIGS. 1 and 2, and garments with sensors are illustrated in FIG. 5, other form factors are also or instead possible, some of which are discussed below by way of example.

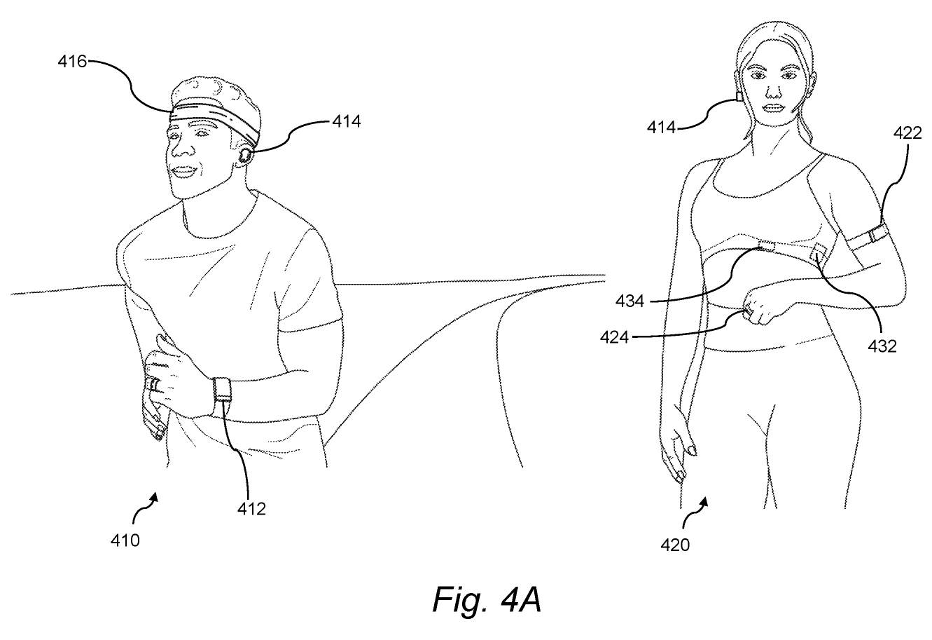

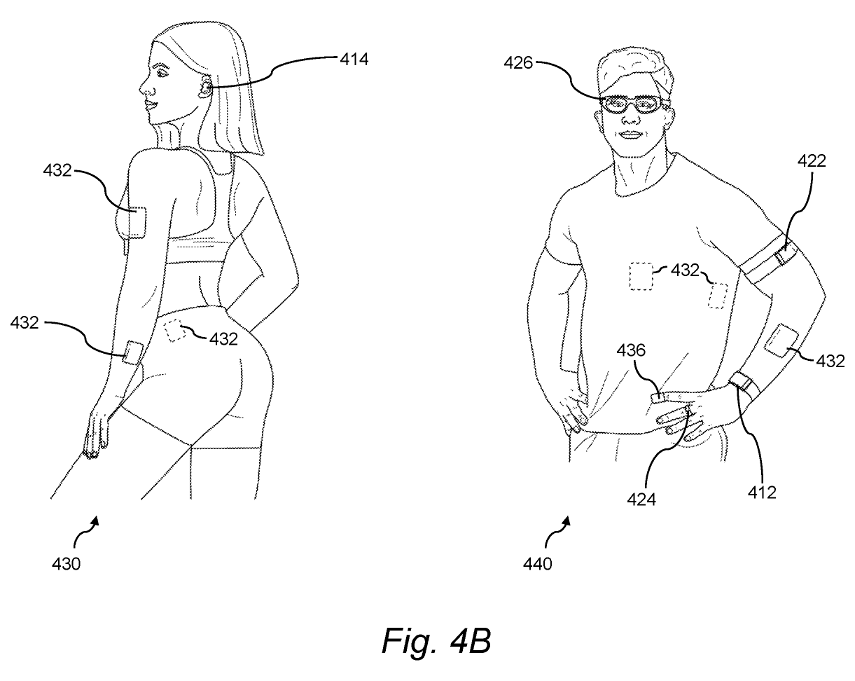

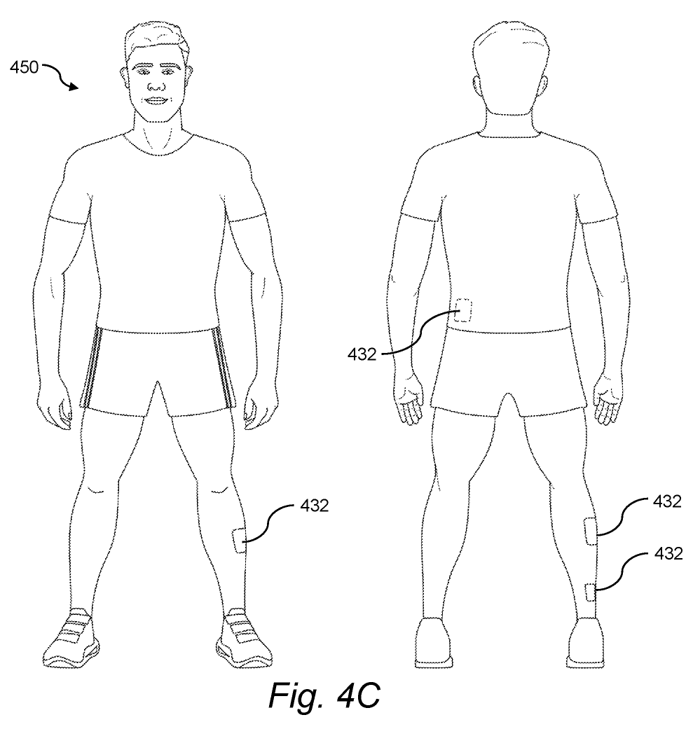

FIGS. 4A–4C illustrate physiological monitoring devices. The illustrated devices may include any of the hardware, software, and/or other components described herein for physiological sensing and/or other functions, and may be embodied in various form factors for various use cases. These various form factors may be used individually or as multiple independent or cooperating physiological monitoring devices, and may include two or more devices of the same type (e.g., two wrist-worn devices, two or more patches, and so on), and/or two or more different types of devices. Moreover, other form factors, and combinations thereof, may also or instead be used for physiological monitoring as described herein. It will further be understood that each of the different example form factors shown in these figures or elsewhere herein may include any one or more of the various sensors, emitters, processors, memories, interfaces, power supplies, and/or other processing and control circuitry, including without limitation any of the foregoing described herein, e.g., with reference to FIGS. 1–3 above.

FIG. 4A shows a first user 410 and a second user 420. The first user 410 may be wearing one or more physiological monitors, such as a wrist-worn device 412 (such as any described herein), an ear-worn device 414 (including on-ear devices retained with a clamp, clip, or other mechanism, and/or in-ear devices, such as earbuds or the like, that are retained at least in part within the ear canal), and a headband 416 or similar.

In one aspect, an ear-worn device 414 may be structurally configured to be partially or entirely inserted within an ear canal of the first user 410. In another aspect, the ear-worn device 414 may be configured to be worn on the ear lobe, or in some other location on the ear where, e.g., temperature, blood flow, respiration, and/or other physiological parameters can be measured. In one aspect, an ear-worn device 414 may be configured for heart rate monitoring, such as any of the heart rate monitoring described herein. For example, this may include continuous heart rate monitoring with optical sensors based on changes in blood volume beneath the skin. The ear-worn device 414 may also or instead be configured for temperature monitoring. For example, the ear-worn device 414 may include one or more infrared sensors, thermistors, thermocouples, or the like to measure the temperature of the ear canal and/or other surfaces. Surface measurements may also or instead be used to support other inferences about body temperature, heat dissipation, and the like, which may be related to current activity levels, general health, and wellness, and so forth.

In another aspect, the ear-worn device 414, or any of the other devices described herein, may be configured for activity tracking. For example, the ear-worn device 414 may include one or more accelerometers, gyroscopes, Global Positioning System (GPS) sensors, and so forth to detect motion and provide information about physical activity levels. This may, for example, include large-scale motion, such as geographical movement and elevation changes that can be tracked with GPS or the like, or local movement detected by the ear-worn device 414, which may be tracked with multi-axis gyroscopes, multi-axis accelerometers, and so forth. These latter sensors may be used to infer, e.g., steps taken, gait analysis, activity type, activity level, and/or overall movement.

The ear-worn device 414, or any of the other devices described herein, may also or instead be configured for blood pressure monitoring. This may, for example, include techniques based on cardiovascular waveform analysis (e.g., using the shape of a PPG or ECG signal from a single location), pulse transit time (e.g., based on the time difference between waveforms at two or more physical locations on the body with two or more monitors), pulse wave velocity (similar to pulse transit time, but over longer arterial distances), physical pulse monitoring (e.g., with pressure sensors, haptic stimulus responses, or other mechanical and/or dynamic techniques), tonometry (measuring the force required to counteract arterial pressure), oscillometric measurement (measuring oscillations in the arterial wall as a cuff deflates around a region of interest), volume clamping (measuring changes in pressure that are required to maintain constant blood volume in a region of interest), and so forth. Some of these blood pressure monitoring techniques are better suited to specific types and locations of monitors and may be more suited to, e.g., wristbands, bicep bands, chest straps, finger rings, and so forth, but are included here for completeness.

The ear-worn device 414, or any of the other devices described herein, may also or instead be configured for electrodermal activity (EDA) monitoring. For example, the ear-worn device 414 may include one or more electrodes in contact with the skin, which may be used to measure the electrical conductance thereof, and to infer, e.g., sweat levels, skin hydration, and/or other parameters correlated to skin conductance. Electrodes may also or instead be used for, e.g., ECG monitoring or the like.

The ear-worn device 414, or any of the other devices described herein, may also or instead be configured to sense blood oxygen saturation (also referred to as pulse oximetry or SpO2) monitoring. To this end, the ear-worn device 414 may include one or more optical sources and detectors, and the system may use different absorption spectra of oxygenated and deoxygenated hemoglobin to estimate pulse oxygen saturation. In another aspect, the ear-worn device 414, or any of the other devices described herein, may be configured for brainwave monitoring, e.g., using electroencephalogram (EEG) sensors to monitor brainwave activity.

The ear-worn device 414, or any of the other devices described herein, may also or instead be configured for respiration rate monitoring. In one aspect, respiration rate may be inferred using respiratory sinus arrhythmia or other techniques to infer respiration rate from a measured heart rate signal over time. In another aspect, respiration rate may be inferred from physical changes in the ear canal (or chest, or other body part, where applicable to a particular sensor). Other techniques may also or instead be used. For example, the ear-worn device 414 may include a microphone or other audio transducer, and the respiration rate may be inferred from audio data acquired from the user.

In another aspect, a headband 416 may be structurally and programmatically configured for physiological sensing and/or monitoring using any of the systems and methods described herein. For example, the headband 416 may be configured to monitor heart rate, temperature, brain activity, electromyography, galvanic skin response, motion, activity, and so forth. In general, the sensors and processing may be adapted for the form factor of the headband 416. For example, the headband 416 may use temperature sensors to measure skin temperature and/or ambient temperature around the head. For brain activity, the headband 416 may include EEG sensors or the like embedded within the headband 416 to measure electrical activity in the brain, which can be used for monitoring brain waves associated with different states such as relaxation, concentration, and/or sleep. More generally, any physiological monitoring techniques described herein that can be adapted for use in a corresponding form factor may be deployed, either alone or in combination, for physiological monitoring with the headband 416. In another aspect, the headband 416 may incorporate a brain-computer interface (BCI) for control of a physiological monitoring system. This may, for example, include any system suitable for direct communication between the brain and external devices based on, e.g., signal acquisition using techniques such as electroencephalography, processing of these raw signals, feature extraction and translation, and then command execution based on an inferred user intention.

The second user 420 may be wearing one or more physiological monitors such as an ear-worn device 414 (which may be any as described herein, and which may be configured as a clamp, clip, earring, or similar, as shown), a bicep band 422, a ring 424, a patch 432 (such as any as described herein, e.g., with reference to FIG. 4B), and a band sensor 434.

The bicep band 422 may be configured for physiological monitoring and sensing using any of the systems and methods described herein, e.g., by retaining a sensor in place with the bicep band 422 or integrating components of the sensor into the bicep band 422, or some combination of these. The bicep band 422 may be configured to monitor heart rate, motion, activity, temperature, blood pressure, blood oxygen saturation, hydration, body composition, ultraviolet light exposure, electrodermal activity, and so forth, as well as combinations of the foregoing. In one aspect, electromyography (EMG) may be used to measure electrical activity in the muscles, e.g., with one or more electrical contacts or the like embedded in the bicep band 422, which can provide information about muscle contraction and fatigue during physical activity. Body composition analysis may be performed using, e.g., bioelectrical impedance analysis to estimate various components of body composition such as fat (percentage or mass), muscle (percentage or mass), and hydration. In another aspect, the bicep band 422 may include one or more sensors to measure ambient light, and more specifically, ambient ultraviolet (UV) light. This may be used to monitor UV exposure, and to provide recommendations to the user to meet certain healthy thresholds for, e.g., vitamin D synthesis, mood, and immune function, and/or to provide alerts concerning possible overexposure. In another aspect, the bicep band 422 or other form factors described herein may be adapted for gesture control based on the capture of motion signals and corresponding inferences of user intent. While a bicep band 422 is illustrated, it will be understood that similar bands for other body parts may also or instead be used, such as leg bands (or more specifically, thigh bands, calf bands, ankle bands, etc.), chest bands, abdomen bands, neck bands, wristbands, and so forth.

The ring 424 may be configured for physiological monitoring and sensing using any of the systems and methods described herein. For example, the ring 424 may be configured to monitor heart rate, motion, activity, sleep, temperature, blood pressure, respiration rate, blood oxygen saturation, hydration, UV exposure, and so forth. A ring 424 is also advantageously positioned to capture a wide range of hand motions, and may be configured for gesture control of physiological monitoring and/or related hardware and software. The ring 424 may be configured for wearing on a finger, as shown in the figure, or another portion of a wearer’s body (e.g., a thumb, a toe, and so forth).

The band sensor 434 may be the same or similar to the other monitors described herein and/or any of the bands as described herein. In an aspect, the band sensor 434 may include a monitor inserted into (e.g., placed into a pocket or the like), coupled with, embedded within, or the like, a strap or band, e.g., an elastic band in an article of clothing, an accessory, or similar.

FIG. 4B shows a third user 430 and a fourth user 440. The third user 430 may be wearing one or more physiological monitors such as an ear-worn device 414, which may be the same as or similar to any of those described herein, and one or more patches 432 that include sensors and the like to support physiological monitoring. By way of example, a patch 432 may be configured for physiological monitoring and sensing of heart rate monitoring, temperature, activity, motion, blood pressure, blood oxygen saturation, respiration rate, blood glucose, perspiration, hydration, ultraviolet exposure, and so forth, as well as combinations of the foregoing. In one aspect, the patch 432 may include a continuous glucose monitor with a sensor for insertion into fatty tissue under the skin, along with a transmitter to wirelessly transmit glucose data to a smartphone or other device. In another aspect, the patch 432 may include a hydration monitor using, e.g., electrical impedance analysis to measure resistance and reactance of body tissue with a small electrical current, or bioimpedance spectroscopy to measure impedance at various frequencies of electrical current. Hydration monitoring may also or instead use a wearable patch to collect sweat and analyze electrolyte concentrations correlated to hydration. Other techniques for measuring hydration using, e.g., near-infrared spectroscopy or capacitance hygrometry, may also or instead be employed where suitable adaptations can be made to any of the wearable monitors described herein. In another aspect, the patch 432, or any of the other monitors described herein, may be adapted to monitor environmental conditions such as temperature, humidity, air quality, noise, light, and the like that might be used to supplement physiological monitoring when evaluating the condition of a user. In another aspect, the patch 432, or any of the other monitors described herein, may be adapted for electrodermal activity monitoring, e.g., for tracking autonomic nervous system activity, stress, and the like based on galvanic skin response. One or more patches 432 may be coupled to a user in one or more of a plurality of locations on the body, such as those shown on the third user 430—e.g., a portion of an arm (e.g., the upper arm and/or the lower arm), and on or near the gluteus maximus, and similar. Other locations are also or instead possible, such as the chest, the abdomen, the forehead or temples, the wrist, a hand, a finger, a foot, a neck, a backside, the pelvic region, a portion of the back, a portion of a leg, and so forth.

The fourth user 440 may be wearing one or more physiological monitors such as a bicep band 422, a wrist-worn device 412, a ring 424, and a patch 432, which may be the same or similar to any of the monitors described herein. The fourth user 440 further is shown with eyewear 426 and a fingertip monitor 436, as further explained below by way of example.

The eyewear 426 may include sensors or the like in contact areas or similar, such as a temple region, face region (e.g., via the frame or lens), or other head portion of the fourth user 440. For example, the eyewear 426 may be configured for physiological monitoring and sensing of heart rate, temperature, brain activity, motion, activity type, blood pressure, blood oxygen saturation, and so forth, as well as combinations of the foregoing. In one aspect, the eyewear 426 may employ electrooculography (EOG) to measure electrical activity of the muscles around the eyes or another region of the head/face, which can be used, e.g., to track eye movements and provide insights into cognitive states, attention levels, fatigue, and so forth. In another aspect, one or more EEG sensors may be integrated into the frame and/or temples of the eyewear 426 to measure electrical activity in the brain. The eyewear 426 may also or instead be configured to perform eye tracking using cameras and/or infrared or other sensors to monitor movement of the eyes, which can be used for various applications, including human-computer interaction, attention monitoring, and so forth. The eyewear 426 may also or instead be configured for augmented reality (AR) and virtual reality (VR) biometrics, e.g., where the eyewear 426 can include sensors that monitor physiological parameters to enhance user experience and safety, and to visually present information to the user related to any of the foregoing. In another aspect, the eyewear 426 may include cameras, microphones, and the like for recording and tracking environment information.

The fingertip monitor 436 may include a clamp, clip, or the like, and may be the same or similar to any of the physiological monitors described herein. In some aspects, the fingertip monitor 436 may include a pulse oximeter configured to measure oxygen saturation and/or heart rate for monitoring respiratory and/or cardiovascular health.

FIG. 4C shows the front and back of a fifth user 450 showing further example locations for a patch 432 or the like as described herein.

More generally, any one or more of the sensing modalities described herein may, provided suitable adaptations can be made, be deployed in any one or more of the wearable devices described herein. Furthermore, one or more of the wearable devices may communicate with one or more other wearable devices and/or with a control device such as a smartphone or other computing device, to perform cooperative monitoring. For example, various monitoring techniques, such as electrocardiography or blood pressure measurements using pulse transit time, may usefully be performed by combining signals from sensors at two or more different body locations, and a control device may usefully acquire signals from multiple devices and locations to perform such analysis. Similarly, multiple motion signals from different body locations may be used to refine activity detection, measure body temperature, and so forth. Thus, in one aspect, two or more wearable devices may cooperate with one another to perform an integrated sensing operation such as any of those described herein.

In another aspect, any one or more of the wearable electronic devices described herein may use energy harvesting to generate power from various external sources, and/or to supplement power supplied by an internal battery or the like. For example, a device may use solar energy harvesting to extract solar energy from ambient light sources. This may include integrating solar cells or other ambient light collectors into the wearable device to capture energy from sunlight and/or artificial light sources. In another aspect, the device may use kinetic energy harvesting to generate energy from movements by a user of the device. In another aspect, the device may use thermal energy harvesting to generate power based on differences between the body of the wearer and the surrounding environment. The device may also or instead use vibration energy harvesting, radio frequency energy harvesting (e.g., by capturing ambient RF signals, such as Wi-Fi or cellular signals, and converting them into usable electrical power), ambient light harvesting, and so forth. Other techniques may also or instead be used to provide external power, such as beam steering or resonant techniques for short-range or medium-range radio frequency power transfers. More generally, any technique or combination of techniques for powering a device, and/or for supplementing an internal power source such as a battery, with power from ambient sources may be used to power one of the monitoring devices described herein.

FIG. 5 shows a smart garment system. One limitation on wearable sensors can be body placement. Devices are typically wrist-based, and may occupy a location that a user would prefer to reserve for other devices or jewelry, or that a user would prefer to leave unadorned for aesthetic or functional reasons. This location also places constraints on what measurements can be taken, and may also limit user activities. For example, a user may be prevented from wearing boxing gloves while wearing a sensing device on their wrist. To address this issue, physiological monitors may also or instead be embedded in clothing, which may be specifically adapted for physiological monitoring with the addition of communications interfaces, power supplies, device location sensors, environmental sensors, geolocation hardware, payment processing systems, and any other components to provide infrastructure and augmentation for wearable physiological monitors. Such “smart garments” offer additional space on a user’s body for supporting monitoring hardware, and may further enable sensing techniques that cannot be achieved with single sensing devices. For example, embedding a plurality of physiological sensors or other electronic/communication devices in a shirt may allow electrical sensors to be placed around a torso to support electrocardiogram (ECG) based heart rate measurements, or placed around muscles such as the pectoralis major, latissimus dorsi, biceps brachii, and other major muscle groups to support muscle oxygen saturation measurements. In another aspect, optical sensors may be positioned along an arterial pathway or the like to support pulse transit time measurements for calculation of blood pressure. The infrastructure provided by a garment may also support other supplemental functions beyond physiological monitoring. For example, wireless antennas may be placed above the upper portion of the thoracic spine to achieve desired communications signals, or a contactless payment system may be embedded in a sleeve cuff for interactions with a payment terminal. Smart garments may also free up body surfaces for other devices. For example, if sensors in a wrist-worn device that provide heart rate monitoring and step counting can be instead embedded in a user’s undergarments, the user may still receive the biometric information they desire, while also being able to wear jewelry or other accessories for suitable occasions.

The present disclosure generally includes smart garment systems and techniques. It will be understood that a “smart garment” as described herein generally includes a garment that incorporates infrastructure and devices to support, augment, or complement various physiological monitoring modes. Such a garment may include a wired, local communication bus for intra-garment hardware communications, a wireless communication system for intra-garment hardware communications, a wireless communication system for extra-garment communications, and so forth. The garment may also or instead include a power supply, a power management system, processing hardware, data storage, and so forth, any of which may support enriched functions for the smart garment.

In general, the smart garment system 500 illustrated in FIG. 5 may include a plurality of components—e.g., a garment 510, one or more modules 520, a controller 530, a processor 540, a memory 542, and so on—capable of communicating with one another over a data network 502. The garment 510 may be wearable by a user 501 and configured to communicate with a module 520 having a physiological sensor 522 that is structurally configured to sense a physiological parameter of the user 501. As discussed herein, the module 520 may be controllable by the controller 530 based at least in part on a location 516 where the module 520 is located on or within the garment 510. This position-based information may be derived from an interaction and/or communication between the module 520 and the garment 510 using various techniques. It will be understood that, while two controllers 530 are shown, the garment 510 may include a single inter-garment controller, or any number of separate controllers 530 in any number of garments 510 (e.g., one per garment, or one for all garments worn by a person, etc.), and/or controllers may be integrated into other modules 520.

For communication over the data network 502, the system 500 may include a network interface 504, which may be integrated into the garment 510, included in the controller 530, or in some other module or component of the system 500, or some combination of these. The network interface 504 may generally include any combination of hardware and software configured to wirelessly communicate data to remote resources. For example, the network interface 504 may use a local connection to a laptop, smartphone, or the like that couples, in turn, to a wide area network for accessing, e.g., web-based or other network-accessible resources. The network interface 504 may also or instead be configured to couple to a local access point such as a router or wireless access point for connecting to the data network 502. In another aspect, the network interface 504 may be a cellular communications data connection for direct, wireless connection to a cellular network or the like.

The data network 502 may be any as described herein. By way of example, some embodiments of the system 500 may be configured to stream information wirelessly to a social network, a data center, a cloud service, and so forth. In some embodiments, data streamed from the system 500 to the data network 502 may be accessed by the user 501 (or other users) via a website. The network interface 504 may thus be configured such that data collected by the system 500 is streamed wirelessly to a remote processing facility 550, database 560, and/or server 570 for processing and access by the user. In some embodiments, data may be transmitted automatically, without user interaction, for example by storing data locally and transmitting the data over available local area network resources when a local access point such as a wireless access point or a relay device (such as a laptop, tablet, or smartphone) is available. In some embodiments, the system 500 may include a cellular system or other hardware for independently accessing network resources from the garment 510 without requiring local network connectivity. It will be understood that the network interface 504 may include a computing device such as a mobile phone or the like. The network interface 504 may also or instead include or be included on another component of the system 500, or some combination of these. Where battery power or communications resources can advantageously be conserved, the system 500 may preferentially use local networking resources when available, and reserve cellular communications for situations where a data storage capacity of the garment 510 is reaching capacity. Thus, for example, the garment 510 may store data locally up to some predetermined threshold for local data storage, below which data is transmitted over local networks when available. The garment 510 may also transmit data to a central resource using a cellular data network only when local storage of data exceeds the predetermined threshold.

The garment 510 may include one or more designated areas 512 for positioning a module to sense a physiological parameter of the user 501 wearing the garment 510. One or more of the designated areas 512 may be specifically tailored for receiving a module 520 therein or thereon. For example, a designated area 512 may include a pocket structurally configured to receive a module 520 therein. Also or instead, a designated area 512 may include a first fastener configured to cooperate with a second fastener disposed on a module 520. One or more of the first fastener and the second fastener may include at least one of a hook-and-loop fastener, a button, a clamp, a clip, a snap, a projection, and a void.

By placing a pocket or the like in one of these designated areas 512, a position of a module 520 can be controlled, and where an RFID tag, sensor, or the like is used, the designated area 512 can specifically sense when a module 520 is positioned there for monitoring, and can communicate the detected location to any suitable control circuitry.

The garment 510 may also or instead incorporate other infrastructure 515 to cooperate with a module 520. For example, the garment infrastructure 515 may include infrastructure 515 related to ECG devices, such as ECG pads (or otherwise electrically conductive sensor pads and/or electrodes that connect to the module 520, controller 530, and/or another component of the system 500), lead wires, and the like. By way of further example, the garment infrastructure 515 may include wires or the like embedded in the garment 510 to facilitate wired data or power transfer between installed modules 520 and other system components (including other modules 520). The infrastructure 515 may also or instead include integrated features for, e.g., powering modules, supporting data communications among modules, and otherwise supporting operation of the system 500. The infrastructure 515 may also or instead include location or identification tags or hardware, a power supply for powering modules 520 or other hardware, communications infrastructure as described herein, a wired intra-garment network, or supplemental components such as a processor, a Global Positioning System (GPS), a timing device, e.g., for synchronizing signals from multiple garments, a beacon for synchronizing signals among multiple modules 520, and so forth. More generally, any hardware, software, or combination of these suitable for augmenting operation of the garment 510 and a physiological monitoring system using the garment 510 may be incorporated as infrastructure 515 into the garment 510 as contemplated herein.

The modules 520 may generally be sized and shaped for placement on or within one or more designated areas 512 of the garment 510. For example, in certain implementations, one or more of the modules 520 may be permanently affixed on or within the garment 510. In such instances, the modules 520 may be washable. Also or instead, in certain implementations, one or more of the modules 520 may be removable and replaceable relative to the garment 510. In such instances, the modules 520 need not be washable, although a module 520 may be designed to be washable and/or otherwise durable enough to withstand a prolonged period of engagement with a designated area 512 of the garment 510. A module 520 may be capable of being positioned in more than one of the designated areas 512 of the garment 510. That is, one or more of the plurality of modules 520 may be configured to sense data using a physiological sensor 522 in a plurality of designated areas 512 of the garment 510.

A module 520 may include one or more physiological sensors 522 and a communications interface 524 programmed to transmit data from at least one of the physiological sensors 522. For example, the physiological sensors 522 may include one or more of a heart rate monitor (e.g., one or more PPG sensors or the like), an oxygen monitor (e.g., a pulse oximeter), a blood pressure monitor, a thermometer, an accelerometer, a gyroscope, a position sensor, a Global Positioning System, a clock, a galvanic skin response (GSR) sensor, or any other electrical, acoustic, optical, camera, or other sensor or combination of sensors and the like useful for physiological monitoring, environmental monitoring, or other monitoring as described herein. In one aspect, the physiological sensors 522 may include a conductivity sensor or the like used for electromyography, electrocardiography, electroencephalography, or other physiological sensing based on electrical signals. The data received from the physiological sensors 522 may include at least one of heart rate data and/or similar data related to blood flow (e.g., from PPG sensors), muscle oxygen saturation data, temperature data, movement data, position/location data, environmental data, temporal data, blood pressure data, and so on.

Thus, certain embodiments include one or more physiological sensors 522 configured to provide continuous measurements of heart rate using photoplethysmography or the like. The physiological sensor 522 may include one or more light emitters for emitting light at one or more desired frequencies toward the user’s skin, and one or more light detectors for receiving light reflected from the user’s skin. The light detectors may include a photo-resistor, a phototransistor, a photodiode, and the like. A processor may process optical data from the light detector(s) to calculate a heart rate based on the measured, reflected light. The optical data may be combined with data from one or more motion sensors, e.g., accelerometers and/or gyroscopes, to minimize or eliminate noise in the heart rate signal caused by motion or other artifacts. The physiological sensor 522 may also or instead provide at least one of continuous motion detection, environmental temperature sensing, electrodermal activity (EDA) sensing, galvanic skin response (GSR) sensing, and the like.

The system 500 may include different types of modules 520. For example, a number of different modules 520 may each provide a particular function. Thus, the garment 510 may house one or more of a temperature module, a heart rate/PPG module, a muscle oxygen saturation module, a haptic module, a wireless communication module, or combinations thereof, any of which may be integrated into a single module 520 or deployed in separate modules 520 that can communicate with one another. Some measurements, such as temperature, motion, optical heart rate detection, and the like, may have preferred or fixed locations, and pockets or fixtures within the garment 510 may be adapted to receive specific types of modules 520 at specific locations within the garment 510. For example, motion may preferentially be detected at or near extremities, while heart rate data may preferentially be gathered near major arteries. In another aspect, some measurements, such as temperature, may be measured anywhere, but may preferably be measured at a single location in order to avoid certain calibration issues that might otherwise arise through arbitrary placement.

In another aspect, the system 500 may include two or more modules 520 placed at different locations and configured to perform differential signal analysis. For example, the rate of pulse travel and the degree of attenuation in a cardiac signal may be detected using two or more modules at two or more locations, e.g., at the bicep and wrist of a user, or at other locations similarly positioned along an artery. These multiple measurements support a differential analysis that permits useful inferences about heart strength, pliability of circulatory pathways, blood pressure, and other aspects of the cardiovascular system that may indicate cardiac age, cardiac health, cardiac conditions, and so forth. Similarly, muscle activity detection might be measured at different locations to facilitate a differential analysis for identifying activity types, determining muscular fitness, and so forth. More generally, multiple sensors can facilitate differential analysis. To facilitate this type of analysis with greater precision, the garment infrastructure may include a beacon or clock for synchronizing signals among multiple modules, particularly where data is temporarily stored locally at each module, or where the data is transmitted to a processor from different locations wirelessly, where packet loss, latency, and the like may present challenges to real-time processing.

The communications interface 524 may be any as described herein, for example, including any of the features of the network interface 504 described above.

The controller 530 may be configured, e.g., by computer-executable code or the like, to determine a location of the module 520. This may be based on contextual measurements, such as accelerometer data from the module 520, which may be analyzed by a machine learning model or the like to infer a body position. In another aspect, this may be based on other signals from the module 520. For example, signals from sensors, such as photodiodes, temperature sensors, resistors, capacitors, and the like, may be used alone or in combination to infer a body position. In another aspect, the location may be determined based on the proximity of a module 520 to a proximity sensor, RFID tag, or the like at or near one of the designated areas 512 of the garment 510. Based on the location, the controller 530 may adapt operation of the module 520 for location-specific operation. This may include selecting filters, processing models, physiological signal detections, and the like. It will be understood that operations of the controller 530, which may be any controller, microcontroller, microprocessor, or other processing circuitry, or the like, may be performed in cooperation with another component of the system 500, such as the processor 540 described herein, one or more of the modules 520, or another computing device. It will also be understood that the controller 530 may be located on a local component of the system 500 (e.g., on the garment 510, in a module 520, and so on) or as part of a remote processing facility 550, or some combination of these. Thus, in an aspect, a controller 530 is included in at least one of the plurality of modules 520. And, in another aspect, the controller 530 is a separate component of the garment 510, and serves to integrate functions of the various modules 520 connected thereto. The controller 530 may also or instead be remote relative to each of the plurality of modules 520, or some combination of these.

The controller 530 may be configured to control one or more of (i) sensing performed by a physiological sensor 522 of the module 520 and (ii) processing by the module 520 of the data received from a physiological sensor 522. That is, in certain aspects, the combination of sensors in the module 520 may vary based on where it is intended to be located on a garment 510. In another aspect, processing of data from a module 520 may vary based on where it is located on a garment 510. In this latter aspect, a processing resource such as the controller 530 or some other local or remote processing resource coupled to the module 520 may detect the location and adapt processing of data from the module 520 based on the location. This may, for example, include a selection of different models, algorithms, or parameters for processing sensed data.

In another aspect, this may include selecting from among a variety of different activity recognition models based on the detected location. For example, a variety of different activity recognition models may be developed, such as machine learning models, lookup tables, analytical models, or the like, which may be applied to accelerometer data to detect an activity type. Other motion data, such as gyroscope data, may also or instead be used, and activity recognition processes may also be augmented by other potentially relevant data, such as data from a barometer, magnetometer, GPS, and so forth. This may generally discriminate, e.g., between being asleep, at rest, or in motion, or this may discriminate more finely among different types of athletic activity, such as walking, running, biking, swimming, playing tennis, playing squash, and so forth. While useful models may be developed for detecting activities in this manner, the nature of the detection will depend upon where the accelerometers are located on a body. Thus, a processing resource may usefully identify location first using location detection systems (such as tags, electromechanical bus connections, etc.) built into the garment 510, and then use this detected location to select a suitable model for activity recognition. This technique may similarly be applied to calibration models, physiological signal processing models, and the like, or to otherwise adapt processing of signals from a module 520 based on the location of the module 520. In general, determining a location of a module 520 may include, e.g., receiving a sensed location for the module 520, determining the location based on communications between the module 520 and the garment 510, determining the location based on data received from a physiological sensor 522 of the module 520, and so forth.

Once determined using any of the techniques above, the location of a module 520 may be transmitted for storage and analysis to a remote processing facility 550, a database 560, or the like. That is, in addition to the module 520 using this information locally to configure itself for the location in which it is worn, the module 520 may communicate this information to other modules 520, peripherals, or the cloud. Processing this information in the cloud may help an organization determine if a module 520 has ever been installed on a garment 510, which locations are most used, and how modules 520 perform differently in different locations. These analytics may be useful for many purposes and may, for example, be used to improve the design or use of modules 520 and garments 510, either for a population, for a user type, or for a particular user.

As stated above, the system 500 may further include a processor 540 and a memory 542. In general, the memory 542 may bear computer-executable code configured to be executed by the processor 540 to perform processing of the data received from one or more modules 520. One or more of the processor 540 and the memory 542 may be located on a local component of the system 500 (e.g., the garment 510, a module 520, the controller 530, and the like) or as part of a remote processing facility 550 or the like, as shown in the figure. Thus, in an aspect, one or more of the processor 540 and the memory 542 are included on at least one of the plurality of modules 520. In this manner, processing may be performed on a central module or on each module 520 independently. In another aspect, one or more of the processor 540 and the memory 542 are remote relative to each of the plurality of modules 520. For example, processing may be performed on a connected peripheral device, such as a smartphone, laptop, local computer, or cloud resource.

The processor 540 may be configured to assess the quality of the data received from a physiological sensor 522 of the module 520, or otherwise process data as described herein. The memory 542 may store one or more algorithms, models, and supporting data (e.g., parameters, calibration results, user selections, and so forth) and the like for transforming data received from a physiological sensor 522 of the module 520. In this manner, suitable models, algorithms, tuning parameters, and the like may be selected for use in transforming the data based on the location of the module 520 as determined by the controller 530 and/or processor 540 as described herein.

A database 560 may be located remotely and in communication with the system 500 via the data network 502. The database 560 may store data related to the system 500 such as any discussed herein—e.g., sensed data, processed data, transformed data, metadata, physiological signal processing models and algorithms, personal activity history, and the like. The system 500 may further include one or more servers 570 that host data, provide a user interface, process data, and so forth in order to facilitate use of the modules 520 and garments 510 as described herein.

It will be appreciated that the garment 510, modules 520, and accompanying garment infrastructure and remote networking/processing resources may advantageously be used in combination to improve physiological monitoring and achieve modes of monitoring not previously available.

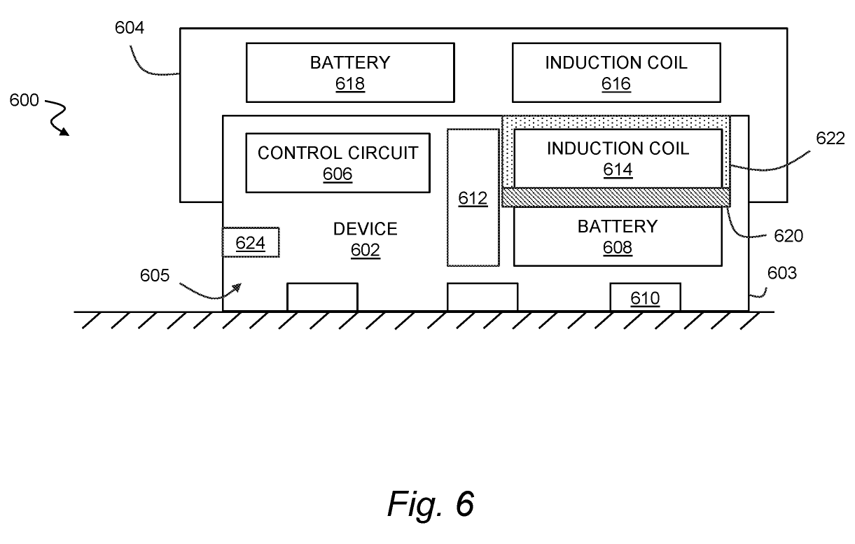

FIG. 6 shows a system 600 for battery pre-heating including a device 602 and a charger 604. In general, increasing a temperature of a rechargeable battery to a typical operating temperature range may improve operation of the rechargeable battery. At low temperatures, electrochemical reaction rates within a rechargeable battery may be reduced, interfering with charging or discharging of the battery. For example, internal impedance may increase, which can produce increased voltage sag, reduced deliverable power, reduced usable capacity, or premature triggering of a low-voltage cutoff during operation of a device. Lithium-ion (Li-ion) and Nickel-Metal Hydride (NiMH) batteries experience reduced charging and/or output capacity at low temperatures. Performance can begin to degrade moderately as soon as temperatures dip below room temperature (< 20 degrees C), with significant reduction in output starting at freezing temperatures (0 degrees C) and severe drops (e.g., 50% reduction in capacity) occurring at -20 degrees C and below. While the target operating range for a battery for charging and discharging will depend on battery chemistry, configuration, expected loads, and so forth, the operation of many rechargeable battery types used in consumer electronics can be improved by preheating, particularly in cold weather. By thermally conditioning a rechargeable battery before or during use, a preheating system can reduce internal resistance, improve charge-carrier mobility, increase voltage stability under load, increase available energy over the discharge interval, and allow the rechargeable battery to support higher-power sensing, processing, communication, or higher charging rates without an unplanned shutdown. Thus, in many thermal conditions, a portion of the energy budget for a battery-powered device can be productively dedicated to creating thermal operating conditions that improve the operation of the battery itself. As described herein, a control circuit can advantageously use preexisting components for a consumer device, such as a wearable fitness monitor, to generate heat that shifts an internal rechargeable battery toward a temperature range in which charge/discharge efficiency, power delivery capability, or runtime of the device is improved.

The device 602, which may be any of the wearable physiological monitoring devices described herein or any other consumer electronic device or the like that runs on battery power, contains a control circuit 606 and a battery 608 such as any of the rechargeable batteries described herein. In general, the device 602 may include a housing 603 and physiological monitoring hardware 605 (or other hardware for specific device purposes) such as processors, sensors, input/output hardware, communications hardware, and any of the other physiological monitoring hardware described herein. Hardware such as the LED 610 may be positioned at various locations within the device 602 for use in sensing, display, and so forth. In general, components of the device 602 such as the first induction coil 614 or the LED 610 may be operated to generate heat in order to precondition the battery 608 for operation of the device 602 or otherwise thermally regulate the device 602. The control circuit 606 may include a processor, microprocessor, microcontroller, or other programmable circuit that executes instructions, e.g., stored in a memory of the device 602 to control operation of the device 602 (including the thermal conditioning processes described herein), as well as any discrete analog circuitry, digital circuitry, or other circuitry suitable for performing the various processing tasks described herein. It will be understood that the device 602 may also include computer-executable instructions, e.g., stored in a memory, that configure the control circuit 606 to implement thermal conditioning processes described herein on the device 602.

The device 602 may include a temperature sensor 612 for monitoring temperature conditions. This may, for example, include a thermistor or other temperature sensing sensor(s), which may be used to monitor a temperature of the battery 608, a temperature of the first induction coil 614, a temperature of the LED 610, an ambient temperature, an interior temperature of the device, or some other temperature associated with the battery 608, or some combination of these.

The LED 610 may be used as a heat source to thermally precondition the battery 608 in cold conditions. The LED 610 may, for example, include a part of an optical sensing system for the device 602, and may be positioned to provide illumination of a target surface such as a user’s skin to facilitate physiological monitoring as described herein. The LED 610 may also or instead be a user interface component for the device, or any other source of light that generates parasitic heat that might be used to thermally precondition the battery 608.

A first induction coil 614 positioned within the device 602 may also or instead be used as a heat source to thermally precondition the battery 608 in cold conditions. The first induction coil 614 may include any coil structure suitable for wireless power transfer, near-field communication, or both, e.g., for wire and/or data transfer between the device 602 and the charger 604. For example, the first induction coil 614 may be implemented as a planar spiral coil, a multi-turn loop coil, a printed conductive trace on a printed circuit board, a printed conductive trace on a flexible circuit substrate, a stamped or etched conductive foil coil, or a wire-wound coil. The first induction coil 614 may include copper, aluminum, silver, or another conductive material, and may be formed as a single-layer coil or as a multi-layer coil having conductive turns on two or more layers that are electrically coupled by vias or other interconnects. In some implementations, the first induction coil 614 may be configured as a near-field communication (NFC) coil, such as a loop antenna configured for operation at about 13.56 MHz, and may be used for wireless transfer of power, wireless transfer of data, or both.

An induction coil or LED may advantageously be used as heat sources for preconditioning because they generate parasitic heat during operation that would otherwise be wasted, and they are both already present in typical embodiments of a wirelessly rechargeable wearable fitness monitor or the like. However, the device 602 may also or instead use parasitic heat from one or more other components of the device 602. For example, the control circuit 606 may include a processor, microcontroller, application-specific integrated circuit, or the like that may be operated at higher clock speeds or used to execute functions or loops with higher processor utilization in order to increase heat output. Other components such as communications hardware, sensing components or systems, haptic outputs, displays, power conversion circuitry, and so forth may also or instead be used where heat output can be controllably initiated or managed by the control circuit 606.

As a further advantage, any of these heat-generating components may be powered by the external recharging battery 618, a USB power source, or any other external power source, thus permitting the battery 608 to be thermally conditioned using power from other sources before or while initiating a charging current to the battery 608. Similarly, the first induction coil 614 may be energized wirelessly from the second induction coil, and a power circuit within the device 602 may convert this into local power that is used to drive one or more components of the device 602, such as an LED, to warm the battery 608 before power from the power circuit is provided to the battery 608, e.g., for charging or the like. More generally, a variety of techniques may be used to convert electricity into thermal energy to pre-condition the battery 608 for charge or discharge as described herein.

The charger 604, which may be any of the replaceable or recharging batteries described herein, may include a second induction coil 616 and a recharging battery 618. In general, the second induction coil 616 may be tuned or matched to the first induction coil 614 to facilitate wireless power and/or data transmission therebetween. Thus, in various aspects, the second induction coil 616 may be controlled (remotely) by the control circuit 606 to generate heat to thermally condition the battery 608 through the housing of the device 602, or to transmit power to the first induction coil 614 for thermal heating thereof. More generally, the second induction coil 616 and/or the recharging battery 618 may be used to support thermal conditioning of the battery 608 as described herein. In use, the charger 604 may be positioned relative to the device 602, e.g., via any suitable removable and replaceable attachment mechanism, to enable inductive coupling when the charger 604 is connected to the device 602, e.g., for charging and/or thermal conditioning as described herein. The second induction coil 616 may, for example, be an NFC coil or other coil(s) for wireless transmission of power and/or data between the charger 604 and the device 602.

In one aspect, one or more of the components of the device 602, such as the LED 610 or the first induction coil 614, may be positioned proximal to, or in thermal contact with, the battery 608 to facilitate heat transfer during preconditioning. Thermal potting material or the like may be used between the battery 608 and a heating device to improve heat transfer. For example, the first induction coil 614 may be located in proximity to the battery 608 to enable efficient heat transfer during the preconditioning process, with thermal potting material 620 therebetween to encourage heat conduction accordingly. For example, a thermally conductive potting material, such as a thermally conductive epoxy, silicone, polyurethane, acrylic, gel, or elastomer loaded with ceramic, boron nitride, aluminum oxide, aluminum nitride, graphite, or other thermally conductive filler, may be disposed between the first induction coil 614 and the battery 608 to facilitate heat transfer from the first induction coil 614 to the battery 608 during preconditioning. Thermal insulation 622 may also or instead be provided around the first induction coil 614, e.g., around one or more surfaces not adjacent to the battery 608. For example, one or more thermally insulating materials, such as an aerogel, silicone foam, polyurethane foam, polyimide foam, fiberglass layer, ceramic fiber layer, low-thermal-conductivity polymer, or vacuum insulation layer, may be positioned adjacent to one or more surfaces of the first induction coil 614 other than a surface facing the battery 608 to reduce heat loss away from the battery 608. In this manner, a preferential heat path may be created between a heat source such as the first induction coil 614 and the battery 608 to encourage heating of the battery 608 while reducing other thermal losses. The LED 610 may also or instead be placed near or in contact with the battery 608 to provide an additional heat source, and may also or instead use any of the thermal management techniques described herein to preferentially encourage heat flow from the LED 610 toward the battery 608 during use.

In general, the control circuit 606 may be configured to regulate the heating process for thermal preconditioning of the battery 608 and initiate charging or discharging when the battery 608 reaches the predefined optimal temperature range. In some cases, the control circuit 606 may activate either the NFC coil (first induction coil 614) or the LED 610 as a heating element, depending on the specific preconditioning requirements and available power sources. The specific preheating method may depend on battery chemistry, battery capacity, device power budgets, and so forth.

By way of non-limiting examples, for a lithium-ion or lithium-polymer battery, including a lithium cobalt oxide, lithium nickel manganese cobalt oxide, lithium nickel cobalt aluminum oxide, lithium manganese oxide, or lithium iron phosphate battery, the control circuit 606 may thermally condition the battery 608 when a temperature associated with the battery 608 is below a lower discharge temperature threshold and the device 602 is expected to draw power from the battery 608 (or below a lower charging temperature threshold prior to charging). The thermal conditioning profile may include heating the battery 608 to a target discharge temperature range before or during a discharge interval, limiting a discharge current until the temperature associated with the battery 608 reaches the target discharge temperature range, or sequencing operation of one or more heat-generating components so that battery temperature is increased before a high-power physiological monitoring, communication, sensing, processing, or user-interface operation. The control circuit 606 may limit a heating rate, a maximum battery temperature, a discharge current, a charging current, or a permitted duration of the thermal conditioning operation to reduce voltage sag, excess impedance, reduced available capacity, charging inefficiency, or unplanned shutdown during low-temperature charge/discharge.

For a lithium iron phosphate battery, the control circuit 606 may apply a thermal conditioning profile having a different lower discharge temperature threshold (or charge temperature threshold), target discharge temperature range, heating rate, discharge-current limit, or permitted heat source than a thermal conditioning profile used for another lithium-based chemistry. In some implementations, the battery 608 may include a lithium titanate battery, and the control circuit 606 may use a thermal conditioning profile that permits discharge at a lower temperature, uses a lower amount of heating before discharge, or enables a different discharge-current limit based on a low-temperature discharge characteristic of the lithium titanate battery.

For a nickel-metal hydride battery or other rechargeable battery chemistry for consumer devices, the control circuit 606 may use a thermal conditioning profile that differs from a lithium-based thermal conditioning profile. For example, the control circuit 606 may heat the battery 608 to reduce internal resistance before a high-current charging or discharging event, may maintain a reduced charge or discharge current until the battery 608 reaches a target temperature range, or may monitor a temperature rise characteristic associated with the battery chemistry during charge or discharge.

More generally, a thermal conditioning profile may specify one or more of a temperature threshold for initiating thermal conditioning, a target charge or discharge temperature range for ending or reducing thermal conditioning, a maximum permitted temperature, a heating rate, a permitted heat source, a duty cycle of the permitted heat source, a discharge current limit, a charge current limit, a state-of-charge window, a predicted load condition, or a monitoring interval for the temperature sensor 612.

During the preconditioning process, the first induction coil 614 may be energized, e.g., from the charger 604 or from another power source within the device 602, to produce heat without initiating a charging or communication process. This heat generation may be achieved through controlled electromagnetic induction, where the resistance of the first induction coil 614 to the flow of electric current results in the production of thermal energy. The power source for heating the first induction coil 614 may vary depending on the state of the device 602 and available energy sources. For example, when the device 602 is connected to an external power source through a connector 624, such as a USB port, the system 600 may use power received through the connector 624 to energize the first induction coil 614 for heating purposes. This allows for thermal preconditioning without depleting the battery 608 of the device 602. Alternatively, in situations where external power is not available, the system 600 may use power from the battery 608 to activate the first induction coil 614 (or other heat source) for heating to improve efficiency of the battery prior to or in parallel with battery discharge for other device functions. This approach may be used when the battery 608 retains sufficient charge to support the preconditioning process while maintaining essential or relevant device functions.

The use of an induction coil or light emitting diode as a heat source offers several advantages. By repurposing an existing component in a device for heating, the need for additional dedicated heating elements is reduced, permitting a reduction in part count and/or overall device size and complexity. Furthermore, the proximity of the induction coil (or LED) to a battery allows for more direct and efficient heat transfer, permitting reduced energy loss during the preconditioning process. The dual functions of a heat source can also contribute to the overall energy efficiency of the device 602. By using a component that is already integrated into the system 600, e.g., for sensing, charging, or communication purposes, the additional power consumption required for preconditioning may be minimized, potentially extending battery life during use. In another aspect, the control circuit 606 may also modulate the power supplied to a heat source to achieve more specific temperature targets during preconditioning and subsequent charging or use. For example, this may include adjusting the duration and intensity of the heating cycles based on feedback from a temperature sensor, ensuring that the battery reaches and maintains the optimal temperature range for charging or discharging.

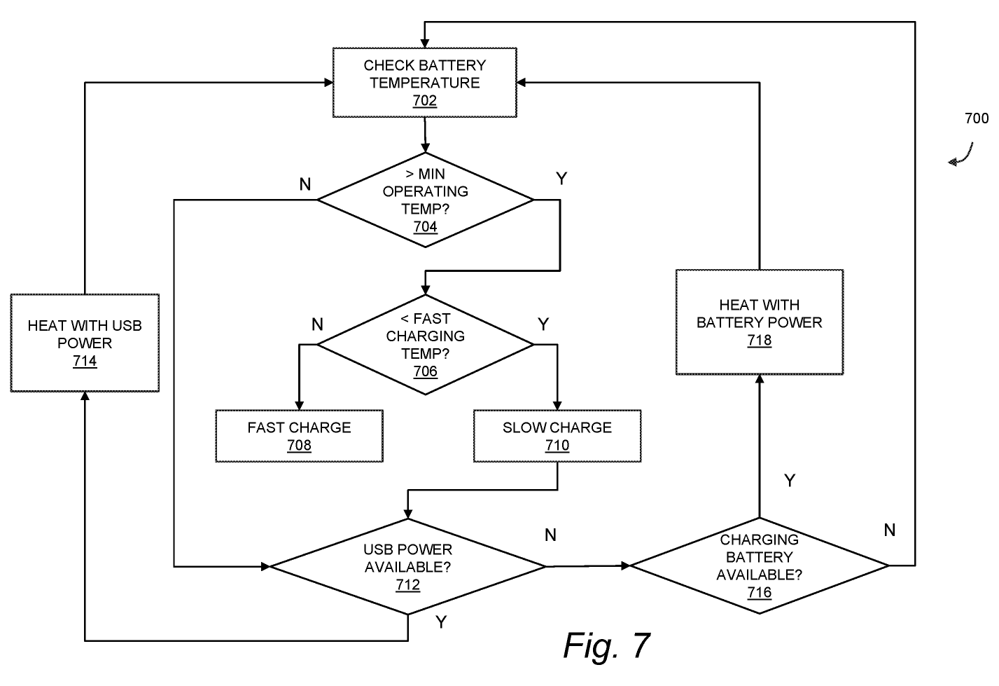

FIG. 7 is a flow chart of a method for thermal conditioning during battery charging. In general, the method 700 may be deployed on any of the devices described herein. In particular, the method 700 may be useful where a device with a rechargeable battery includes a USB port and/or an NFC coil to provide charging to the rechargeable battery from an external source.

As shown in step 702, the method 700 may begin with temperature detection. A temperature sensor, such as any described herein, may continuously monitor the battery temperature (or some proxy for battery temperature such as a nearby internal, external, or ambient temperature) and provide this information to a control circuit. When the control circuit detects that the battery temperature is below a predetermined threshold, the preheating process may be initiated.

As shown in step 704, the method 700 may include determining whether the detected temperature is above a minimum operating temperature. If the temperature is above the minimum operating temperature, the method 700 may proceed to step 706 where it may be determined whether the temperature is below a fast charging temperature for the battery. If the temperature for the battery is above a fast charging threshold, the method 700 may proceed to step 708 where fast charging may be initiated. Fast charging may then continue until a full charge is obtained for the battery, subject to any conditions such as loss of charging power, temperature rising above a temperature limit, or some other condition or combination of conditions for which an exit from the fast charging cycle is appropriate. If the battery temperature is less than the fast charging threshold, e.g., not sufficiently warm for fast and efficient fast charging, then the method may proceed to step 710 where a slow charge may be initiated.

As shown in step 712, once a slow charge has been initiated, the method 700 may determine whether USB power is available. If USB power is available, then the method 700 may proceed to step 714 where the battery may be preheated with USB power provided to an NFC coil, LED, or other component in the device that generates heat during operation. If USB power is not available to operate circuitry of the device, the method may proceed to step 716 where it may be determined whether a charging battery, such as any of the wireless recharging batteries described herein, is available to provide power to the pre-heating process.

As shown in step 716, if a charging battery is available, then the method may proceed to step 718 where the battery may be heated with power from the NFC coil, LED or other component in the device that generates heat. If a charging battery is not available, then the method may return to step 702 where battery temperature may once again be checked prior to further recharging processes. In this manner the system may continuously loop while charging in order to determine whether the temperature is optimal, and whether a power source is available when needed to heat the battery to a temperature where more efficient or faster charging can be performed. More generally, during the preheating process, the control circuit may continuously monitor the battery temperature and adjust the heating process as necessary or helpful to improve charging efficiency while avoiding overheating.

While illustrated as discrete steps, it will be understood that the method 700 may be modified or augmented to more effectively switch between charging states in response to variations in battery temperature and power availability. For example, as the battery temperature approaches a predetermined optimal range, such as at or near the fast charging temperature, the control circuit may begin to reduce the power supplied to the induction coil. While a binary (e.g., setpoint) or proportional control based on instantaneous feedback may be used, a gradual reduction in heating may advantageously help prevent temperature overshoot and ensure that the battery reaches the desired temperature range without exceeding it. Once the battery temperature reaches the predetermined optimal range, and where the charging power is obtained through a USB source, the control circuit may deactivate the induction coil. In some cases, the control circuit may implement a hysteresis control strategy to maintain the battery temperature within the optimal range. This approach may involve reactivating the induction coil for brief periods if the battery temperature drops below a certain threshold, ensuring that the temperature remains within the desired range during charging or discharging operations. The preheating process may also include safety measures to prevent excessive heating. For example, the control circuit may incorporate temperature limits that, if exceeded, trigger an immediate shutdown of the heating process and potentially alert the user to a potential issue.

In some implementations, the preheating process may be adaptive, adjusting its parameters based on factors such as ambient temperature, battery age, or historical preheating data. This adaptability may allow the system to optimize the preheating process over time, potentially improving efficiency and battery longevity. Further, where the power for preheating is provided by an external source such as a recharging battery pack, a user notification may be provided to suggest continued attachment of the recharging battery pack regardless of the state of charge. Thus, for example, where very cold ambient conditions are detected (or predicted, e.g., using a weather feed or the like), and where the user is expected to exercise outdoors, the user may be notified with a suggestion to attach the recharging battery pack prior to outdoor exercise.

Where an LED is used, the LED may be positioned and operated in a manner optimized for battery thermal conditioning. For example, the LED may be configured to generate heat without full illumination, providing an efficient method for localized heating of the battery. The LED may be operated at a low power setting or some other power setting specifically for heat generation purposes. This low power operation, for example, may allow the LED to produce thermal energy without emitting significant visible light. By controlling the current supplied to the LED, the system may regulate the amount of heat generated, advantageously enabling temperature management during the preconditioning process. The LED may also be positioned for localized heating of specific areas of the battery, allowing for targeted temperature control. This localized heating approach may be particularly beneficial in situations where uniform heating across the entire battery is not necessary or when addressing specific cold spots within the battery structure. In some cases, the control circuit may also modulate the power supplied to the LED to achieve precise temperature control during preconditioning, e.g., by adjusting the duration and intensity of the heating cycles based on feedback from the temperature sensor, ensuring that the battery reaches and maintains the optimal temperature range for charging or discharging. The LED may also be used in conjunction with other heating elements, such as an induction coil, to provide a multi-faceted approach to battery preconditioning. In some implementations, the system may selectively activate either the LED or the induction coil based on factors such as available power sources, desired heating rate, or specific temperature requirements. In one aspect, the control circuit may implement a graduated heating process by incrementally increasing power to the selected heating element (e.g., a parasitic heat source) as the temperature of the rechargeable battery increases toward the predetermined temperature range.

For charging operations, the system may aim to maintain the battery within a temperature range that maximizes charging efficiency while minimizing the risk of lithium plating or other detrimental effects associated with low-temperature charging. Conversely, during discharging, the temperature control system may target a slightly different range that optimizes power output and reduces internal resistance. To this end, the control circuit may implement adaptive strategies to achieve and maintain these optimal temperature ranges. For instance, the system may adjust its heating parameters based on the rate of temperature change, ambient conditions, or historical data from previous charging and discharging cycles. This adaptability may allow the system to optimize its performance over time and in varying environmental conditions. In some implementations, the temperature control system may incorporate predictive algorithms to anticipate temperature changes and proactively adjust heating parameters. By analyzing factors such as charging rate, ambient temperature trends, and battery usage patterns, the system may be able to maintain more stable temperatures and potentially reduce the frequency of heating element activation.

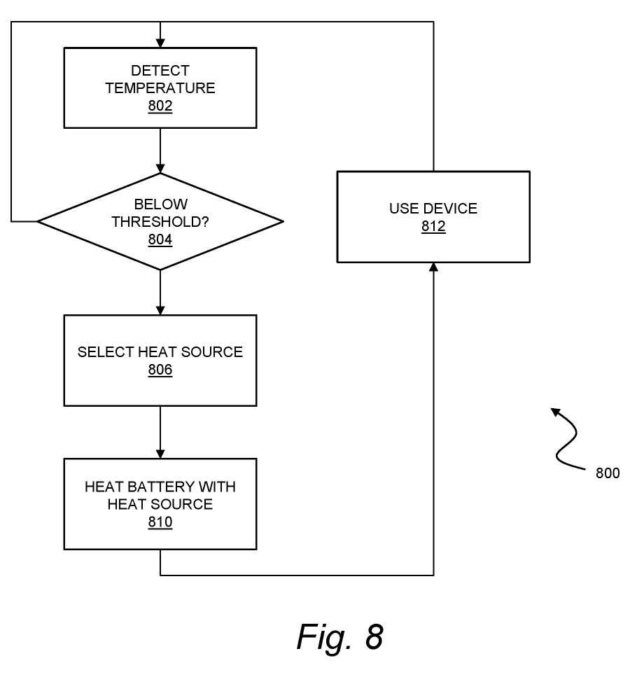

FIG. 8 shows a method 800 for battery preheating. In general, the method 800 may be performed using any of the devices and/or rechargeable batteries described herein, and may be particularly useful in devices where one or more components include a parasitic heat source that generates parasitic heat--unintentional, unwanted heat generated due to non-ideal physical properties, such as stray resistance, capacitance, or inductance, that is unrelated to the intended function of a component—that can be used as a heat source for thermal preconditioning. For example, a function of the parasitic heat source of a wearable device can include one or more of physiological sensing, environmental monitoring, data acquisition, power transfer, and communication with a remote device.

As shown in step 802, the method 800 may begin with detecting a temperature associated with the rechargeable battery, e.g., using a temperature sensor, such as any of the temperature sensors described herein. The temperature may include a temperature of the rechargeable battery, a temperature of a component thermally coupled to the rechargeable battery, an internal temperature of the wearable device, an external temperature of the wearable device, an ambient temperature, or another temperature usable as a proxy for the temperature of the rechargeable battery, and/or the charging or discharging performance of the rechargeable battery.

As shown in step 804, the method 800 may include determining whether the temperature associated with the rechargeable battery fails to meet a predetermined threshold. For example, a control circuit of the wearable device may compare the detected temperature to a lower temperature threshold associated with a desired use of the rechargeable battery. If the detected temperature meets the predetermined threshold, the method 800 may proceed to step 812, where the wearable device is operated using power from the rechargeable battery. If the detected temperature fails to meet the predetermined threshold, the method 800 may proceed to step 806, where a heat source is selected for thermally preconditioning the battery.

As shown in step 806, the method 800 may include selecting a heat source for heating the rechargeable battery. The heat source may include an induction coil, an LED, or another component within the device and adjacent to the rechargeable battery that can provide heat under the control of a control circuit of the device. In some implementations, the wearable device may include both the LED and the induction coil, and the control circuit may select between the LED and the induction coil based on a rate of temperature increase required to bring the rechargeable battery within a predetermined temperature range during a predetermined time period. In some implementations, when the induction coil is selected, the control circuit may energize the induction coil to generate heat without initiating a charging process for the rechargeable battery. In some implementations, when the LED is selected, the control circuit may operate the LED to generate heat for the rechargeable battery and may gradually increase power to the LED as the temperature of the rechargeable battery approaches the predetermined temperature range.

As shown in step 810, the method 800 may include activating the selected heat source to heat the rechargeable battery and monitoring the temperature associated with the rechargeable battery while the rechargeable battery is heated. The control circuit may continue heating until the temperature associated with the rechargeable battery falls within the predetermined temperature range. The control circuit may also reduce power to the selected heat source, deactivate the selected heat source, or select a different heat source based on the monitored temperature, a heating rate, a maximum temperature limit, an available power level, or an expected operating load of the wearable device.

As shown in step 812, the method 800 may include initiating a use of the rechargeable battery after the temperature associated with the rechargeable battery falls within the predetermined temperature range. The use of the rechargeable battery may include operating the wearable device, operating a sensing system of the wearable device, or communicating with a remote device. In this context, using the rechargeable battery may also include recharging the rechargeable battery, e.g., from a remote power source such as any described herein. Using the rechargeable battery may also or instead include operating a sensing system of a wearable device, operating a communications system of a wearable device, or otherwise acquiring data, processing data, or interacting with a user or an external computing device. In general, the control circuit may operate one or more physiological sensors, processing circuits, communication circuits, user-interface components, or other loads of the wearable device using power delivered by the thermally conditioned rechargeable battery.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application-specific integrated circuits, programmable gate arrays, programmable array logic components, or any other device or devices that may be configured to process electronic signals. It will further be appreciated that a realization of the processes or devices described above may include computer-executable code created using a structured programming language such as C, an object-oriented programming language such as C++, or any other high-level or low-level programming language (including assembly languages, hardware description languages, and database programming languages and technologies) that may be stored, compiled, or interpreted to run on one of the above devices, as well as heterogeneous combinations of processors, processor architectures, or combinations of different hardware and software.

Thus, in one aspect, each method described above, and combinations thereof, may be embodied in computer-executable code that, when executing on one or more computing devices, performs the steps thereof. In another aspect, the methods may be embodied in systems that perform the steps thereof, and may be distributed across devices in a number of ways, or all of the functionalities may be integrated into a dedicated, standalone device or other hardware. The code may be stored in a non-transitory fashion in a computer memory, which may be a memory from which the program executes (such as random access memory associated with a processor), or a storage device such as a disk drive, flash memory, or any other optical, electromagnetic, magnetic, infrared, or other device or combination of devices. In another aspect, any of the systems and methods described above may be embodied in any suitable transmission or propagation medium carrying computer-executable code and/or any inputs or outputs from same. In another aspect, means for performing the steps associated with the processes described above may include any of the hardware and/or software described above. All such permutations and combinations are intended to fall within the scope of the present disclosure.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party, such as a remote user, a remote processing resource (e.g., a server or cloud computer), or a machine, to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims.

What is claimed is:

1. A wearable device, comprising:
physiological monitoring hardware;
a housing;
a rechargeable battery disposed within the housing;
an induction coil positioned in the housing proximate to the rechargeable battery;
an LED positioned in the housing proximate to the rechargeable battery;
a temperature sensor configured to monitor a temperature of the rechargeable battery; and
a control circuit operatively connected to the induction coil, the LED, and the temperature sensor, wherein the control circuit is configured to:
select at least one of the induction coil and the LED as a selected heating element to heat the rechargeable battery in response to the temperature of the rechargeable battery failing to meet a predetermined threshold,
operate the selected heating element to increase the temperature of the rechargeable battery, and
initiate a discharge of the rechargeable battery to operate the physiological monitoring hardware of the wearable device for acquisition of physiological data in response to the temperature falling within a predetermined temperature range.

2. The wearable device of claim 1, further comprising a thermally conductive material positioned between at least one of the induction coil or the LED and the rechargeable battery to enhance an efficiency of heat transfer to the rechargeable battery.

3. The wearable device of claim 2, wherein the control circuit is further configured to select between activating the induction coil or the LED based on an available power source in the wearable device.

4. The wearable device of claim 3, wherein the control circuit is configured to activate the induction coil as the selected heating element when external power is available through a charging port and activate the LED as the selected heating element when operating the device from the rechargeable battery.

5. The wearable device of claim 1, wherein the control circuit is configured to implement a graduated heating process by incrementally increasing power to the selected heating element as the temperature of the rechargeable battery increases toward the predetermined temperature range.

6. The wearable device of claim 5, wherein the control circuit is further configured to deactivate the selected heating element upon reaching the predetermined temperature range.

7. A battery preconditioning system for a wearable device, comprising:
a rechargeable battery;
a heating element positioned proximate to the rechargeable battery;
a temperature sensor configured to monitor a temperature of the rechargeable battery; and
a control circuit operatively connected to the heating element and the temperature sensor, wherein the control circuit is configured to:
activate the heating element to heat the rechargeable battery in response to the temperature of the rechargeable battery failing to meet a predetermined threshold, and
initiate charging or discharging of the rechargeable battery in response to the temperature of the rechargeable battery falling within a predetermined temperature range.

8. The battery preconditioning system of claim 7, wherein the heating element comprises an induction coil.

9. The battery preconditioning system of claim 8, wherein the induction coil is configured to generate heat without initiating a charging process.

10. The battery preconditioning system of claim 7, wherein the heating element comprises an LED configured to generate heat at a low power setting.

11. The battery preconditioning system of claim 7, further comprising a thermally conductive material positioned between the heating element and the rechargeable battery to enhance heat transfer efficiency.

12. The battery preconditioning system of claim 7, wherein the control circuit is further configured to deactivate the heating element in response to the temperature reaching the predetermined temperature range.

13. The battery preconditioning system of claim 7, wherein the wearable device includes a wearable fitness monitor.

14. The battery preconditioning system of claim 7, wherein the temperature sensor includes a thermistor.

15. A method of preconditioning a rechargeable battery in a device, the method comprising:
detecting a temperature associated with the rechargeable battery using a temperature sensor;
determining that the temperature fails to meet a predetermined threshold;
in response to the temperature failing to meet the predetermined threshold, activating a heating element of a parasitic heat source proximate to the rechargeable battery to heat the rechargeable battery;
monitoring the temperature as the rechargeable battery is heated with the heating element; and
in response to the temperature falling within a predetermined temperature range, initiating a use of the rechargeable battery.

16. The method of claim 15, wherein the parasitic heat source of the device may have a function on the device including one or more of physiological sensing, environmental monitoring, data acquisition, power transfer, and communications with a remote device.

17. The method of claim 15, wherein the heating element comprises an induction coil, and activating the heating element includes energizing the induction coil to generate heat without initiating a charging process for the rechargeable battery.

18. The method of claim 15, wherein the heating element comprises an LED, and activating the heating element includes operating the LED to generate heat for the rechargeable battery.

19. The method of claim 18, further comprising gradually increasing a power to the LED as the temperature of the rechargeable battery approaches the predetermined temperature range.

20. The method of claim 15, wherein the device includes an LED and an induction coil, the method further comprising selecting between the LED and the induction coil for use as the heating element based on a rate of temperature increase required to reach the predetermined temperature range within a predetermined time period.
